(19) United States
(12) Patent Application Publication
(10) Pub. No.: US 2017/0067894 A1
(43) Pub. Date: Mar. 9, 2017

(54) METHOD AND DEVICE FOR DETECTION OF PSEUDOMONAS AERUGINOSA

(71) Applicants: YISSUM RESEARCH DEVELOPMENT COMPANY OF THE HEBREW UNIVERSITY OF JERUSALEM LTD., Jerusalem (IL); YEDA RESEARCH AND DEVELOPMENT CO. LTD., Rehovot (IL)

(72) Inventors: Yael HELMAN, Tel-Aviv (IL); Noam SOBEL, Jaffa (IL); Sagit SHUSHAN, Rehovot (IL); Igor KVIATKOVSKI, Tel-Aviv (IL); Idan FRUMIN, Rehovot (IL); Leonid CHERNIN, Rehovot (IL); Lavi SECUNDO, Tel Aviv (IL)

(21) Appl. No.: 15/120,772

(22) PCT Filed: Mar. 3, 2015
(86) PCT No.: PCT/IL2015/050227

§ 371 Date: Aug. 23, 2016

Related U.S. Application Data
(60) Provisional application No. 61/947,080, filed on Mar. 3, 2014.

(51) Int. Cl. G01N 33/569; G01N 33/50

(52) U.S. Cl. CPC G01N 33/56911; G01N 33/5091

(57) ABSTRACT

A method of and device for detecting and diagnosing Pseudomonal aeruginosa in a gaseous, liquid or solid sample, employing Lux-R-like receptor-driven reporter cells.

### FIELD AND BACKGROUND OF THE INVENTION

The present invention, in some embodiments thereof, relates to a device and method for detection of Pseudomonas aeruginosa and volatile organic compounds characterizing such.

Pseudomonas aeruginosa is a Gram-negative bacterial pathogen responsible for up to −14% of all nosocomial infections and up to −23% of infections in intensive care units. This bacterium is the most common cause of infections in burn injuries and infections of the outer ear (otitis externa—including malignant otitis externa), as well as the most common respiratory pathogen in cystic fibrosis patients, leading to high rates of morbidity and mortality. P. aeruginosa infections are characterized by high antibiotic resistance and require specific treatment, usually combining two different antibiotics. It is therefore highly important to identify P. aeruginosa infections as early as possible. The most common method used today is culture inoculations, which can identify P. aeruginosa in two days.

P. aeruginosa bacteria produce 2-Aminoacetophenone (2-AA), a volatile substance with a grape-like odor. Using ion-spray gas chromatography analysis several studies showed that 2-AA can serve as a biomarker for P. aeruginosa infections in breath tests of cystic fibrosis patients (Metters et al, Analyst 2014; 16:3999, Scott-Thomas et al BMC Pulm Med 2010; 10:56).

2-AA has been associated with quorum sensing signaling in P. aeruginosa (see, for example Bandyopadhaya et al, PLoS Patholog 2012; On Line Publication November 15), however the signaling pathway(s) and role(s) of 2-AA in P. aeruginosa are poorly understood. 2-AA has no known receptor in P. aeruginosa.

2-AA is one of a large group of bacterial volatile organic compounds (VOC), bacterial metabolites, which have been proven useful for diagnosing a number of diseases and conditions, including diabetes, gastrointestinal and liver disease, lung disorders, some cancers and infections. For example, Reuther et al (US Patent Application US2014/0336081) teaches diagnosis of pneumonia by detection of VOCs characteristic of microbial pneumonia pathogens in a subject's breath, blood or tissue.

Ideally, VOC analysis could eventually eliminate the need for bacterial culture of suspected infection or contamination in order to identify the pathogen. To date, analysis of VOC in biological or non-biological (medical instrumentation, soil, water, etc) samples is mostly based on Gas Chromatography (GS), Mass Spectrometry (MS), combined GC-MS, chemiluminescence, optical absorption spectroscopy systems, “electronic noses” and gaseous sensor systems. Specialized devices may be specifically effective for detecting certain VOCs: e.g. flame ionization detection gas chromatography (GC-FID), proton transfer reaction mass spectrometry (PTR-MS).

Multidimensional sensor arrays, consisting of dedicated or non-selective sensors that interact with VOCs to create a physical or chemical change which sends a signal output (optical, electronic) to a computer, can create detailed profiles of sample components, which can then be correlated to the presence of or absence of disease. Such “electronic noses”, though, are complex, costly and require complex computing power to characterize the sample profiles. Biosensors recognizing a specified ligand, a biosensor for detecting binding of the ligand and means for reporting the binding of the ligand constitute attempts to provide biologically-based sensor systems for component profiling. US Patent Application 2004/019932 to Hseih et al. describes the fabrication and use of an “artificial nose” combining biological ligand binding with piezo-based sensitivity. Generalized detection systems utilizing hybridoma cells as biosensors are also taught in US Patent Application 2014/0273020 to Zupancic et al.

Sayler et al (US Patent Application 2003/0027241) discloses genetically engineered bacteriophages and bioluminescent bioreporter cells which emit light upon infection of target microorganisms by the bacteriophages. Bioluminescence is produced via the reporter cell's Vibrio LuxR-luxCDABE construct in response to Lux-I-carrying bacteriophage infection of target bacterium.

Michelini et al (Biosensors & Bioelectronics 2005, 20, 2261-2267) and Leskinen et al. (Chemosphere 2005, 61, 259-266) describe a bioluminescent assay for the detection of compounds with androgenic activity using recombinant S. cerevisiae. Mirasoli et al (Anal. Chem. 2002, 74, 5948-5953) describe a bacterial biosensor with an internal signal correction. D'Souza S F (Microbial biosensors. Biosens Bioelectron. 2001 August; 16(6):337-53) discloses the entrapment of viable cells in polymeric matrices for the manufacture of stable bioluminescent cell biosensors. Simpson M L et al (Trends Biotech, 1998; 16:332-338) describes bioluminescent microbial biosensors, in which cells are encapsulated in a polymeric matrix to increase the biosensor stability.

Taiwanese Patent TW 239392 discloses a portable biosensing system combined with specific signal processing to detect water toxicity or nutritive properties. The system is based on the inhibition of microbial growth due to sample toxicity. US Patent Application US 2008/032326 discloses a water quality analyzer based on an electro-osmosis cell and a plurality of photosynthetic organisms.

PCT Application WO2007083137 discloses a device composed of biosensors able to detect a specific analyte on the basis of the emission of volatile substances, an immobilization procedure is envisaged based on the use of a matrix of agar, agarose and alginate, all components commonly used for the immobilization of bacterial cells. PCT Application WO2008152124 discloses an engineered yeast cell used as biosensor.

US Patent Application US 2003/162164A1 discloses a testing system in which cells are fixed onto a multiwell support by means of a suspension medium.

Nivens et al reported BBIC (Bioluminescent Bioreporter Integrated Circuits) using bacterial liquid cultures (Nivens, D E, J. Appl. Microbiol. 2004; 96(1):33-46).

Additional relevant publications include US2007/0003996 to Hitt et al, Boots et al (J Breath Res. 8, 2014 127106), Winson et al (FEMS Microb Lett 1998 163:185-92), Kereswani et al (PLoS pathogens, 2011, 7:e1002192), Que et al (PLoS 2013 ONE 8: e80140) and Scott-Thomas et al (BMC Pulm Med 2010; 10:56).

### SUMMARY OF THE INVENTION

According to an aspect of some embodiments of the present invention there is provided a device, comprising a reporter cell comprising a polynucleotide comprising a first nucleic acid sequence encoding a reporter molecule capable of producing a detectable signal and a second nucleic acid sequence comprising a luxR-like receptor binding element for regulating transcription of the reporter molecule, wherein the reporter cell is attached to a solid support or encapsulated within an encapsulation matrix attached to a solid support.

According to an aspect of some embodiments of the present invention there is provided a method of detecting 2AA or derivatives thereof in a biological sample, comprising contacting the sample with the device of the invention, wherein presence of the detectable signal is indicative of 2-AA in the biological sample.

According to an aspect of some embodiments of the present invention there is provided a method of diagnosing a Pseudomonas aeruginosa infection in a subject, comprising contacting a biological sample of the subject with the device of the invention, wherein detection of the signal above a predetermined level is indicative of the presence of a Psuedomonas aeruginosa infection in the sample.

According to an aspect of some embodiments of the present invention there is provided a method of detecting the presence of a 2AA-producing organism in a test sample, comprising contacting the sample comprising the organism with the device of the invention, wherein the presence of the detectable signal is indicative of the presence of a 2AA-producing organism in the test sample.

According to an aspect of some embodiments of the present invention there is provided a system for detecting a Pseudomonas aeruginosa infection in a subject, the system comprising the device of the invention and a sensor for detecting the detectable signal.

According to some embodiments of the invention, the cell further comprises a third nucleic acid sequence encoding a luxR-like receptor capable of binding 2AA.

According to some embodiments of the invention, the polynucleotide comprises the first nucleic acid sequence, the second nucleic acid sequence and the third nucleic acid sequence.

According to some embodiments of the invention, the third nucleic acid sequence is comprised on a polynucleotide distinct of the polynucleotide.

According to some embodiments of the invention, the luxR-like receptor is foreign to the cell.

According to some embodiments of the invention, the luxR-like receptor is selected from the group consisting of a Vibrio LuxR-like receptor, a Pectobacterium LuxR-like receptor, a Burkholderia LuxR-like receptor, a Serratia LuxR-like receptor, a Pseudomonas LuxR-like receptor, a Chromobacteria LuxR-like receptor and a Halomonas LuxR-like receptor.

According to some embodiments of the invention, the cell is devoid of endogenous luxR-like expression.

According to some embodiments of the invention, the cell is devoid of endogenous luxR-like agonists or antagonists or both.

According to some embodiments of the invention, the cell is devoid of luxI expression.

According to some embodiments of the invention, the detectable signal is selected from the group consisting of an optical signal, a chemical signal and an electrochemical signal.

According to some embodiments of the invention, the detectable signal is bioluminescence.

According to some embodiments of the invention, the reporter molecule is a reporter polypeptide.

According to some embodiments of the invention, the reporter polypeptide is luciferase.

According to some embodiments of the invention, the first nucleic acid sequence comprises a luxCDABE gene cluster.

According to some embodiments of the invention, the reporter cell is selected from the group consisting of a bacterial cell, a fungal cell, a plant cell, an algal cell and an animal cell.

According to some embodiments of the invention, the reporter cell is a bacterial cell.

According to some embodiments of the invention, the bacterial cell is selected from the group consisting of Escherichia, Pseudomonas, Vibrio, Staphylococcus, Alcaligenes, Acinetobacter, Synechococcus, Aeromonas hydrophila and Ralstonia.

According to some embodiments of the invention, the reporter cell is E. coli.

According to some embodiments of the invention, the reporter cell is positioned within an encapsulation matrix.

According to some embodiments of the invention, the reporter cell is immobilized on a solid support.

According to some embodiments of the invention, the reporter cell is positioned to contact a volatile sample.

According to some embodiments of the invention, the method further comprises calibrating the device with 2 AA standard samples so as to assign amounts or concentrations of 2AA to values of the detectable signal.

According to some embodiments of the invention, the contacting is via fluid communication.

According to some embodiments of the invention, the contacting is via gaseous communication.

According to some embodiments of the invention, the biological sample is a selected from the group consisting of bacterial culture, bacterial culture fluid, respiratory air, sweat, saliva, sputum, blood, plasma, urine, milk (mammary secretion), pleural fluid, cerebrospinal fluid, meningeal fluid, amniotic fluid, lymph, glandular secretions, semen, pus, feces, vomitus, tears, tissue biopsy, cell culture, ambient air, tissue sample obtained from a wound or burn, a swab obtained from the nose, ear and eyes, mouth vagina, wound, burn or any other tissue suspected of having an infection, phlegm and mucus.

According to some embodiments of the invention, the biological sample is a gaseous sample.

According to some embodiments of the invention, the gaseous sample is headspace gas collected from a source selected from the group of consisting of respiratory air, sweat, saliva, sputum, blood, plasma, urine, milk (mammary secretion), pleural fluid, cerebrospinal fluid, meningeal fluid, amniotic fluid, lymph, glandular secretions, semen, pus, feces, vomitus, tears, tissue biopsy, cell culture, ambient air, tissue sample obtained from a wound or burn, a swab obtained from the nose, ear and eyes, mouth vagina, wound, burn or any other tissue suspected of having an infection, phlegm, mucus, exudate or breath of an animal or human patient.

According to some embodiments of the invention, the gaseous sample comprises headspace gas of a suspected bacterial population.

According to some embodiments of the invention, the suspected bacterial population is a suspected bacterial infection.

According to some embodiments of the invention, the 2AA-producing organism is Pseudomonas aeruginosa.

According to some embodiments of the invention, the amount or pattern of the 2AA detected is indicative of a Pseudomonas aeruginosa infection.

According to some embodiments of the invention, the subject is suspected suffering from otitis and the biological sample is a sample of otic exudate, headspace gas collected from the otic exudate or headspace gas collected from the affected ear.

According to some embodiments of the invention, the system further comprises a display for displaying an event of detection of the detectable signal.

According to some embodiments of the invention, the system further comprises a sample holder in fluid or gaseous communication with the reporter cell, for holding a biological sample from the subject for detection.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a graph showing the induction of luminescence in the E. coli/pSB401 reporter strain by volatiles of P. aeruginosa. Relative luminescence of E. coli/pSB401 was measured after overnight exposure to volatile compounds of P. aeruginosa (PA14). Colonies of the reporter strain were scraped from the agar, resuspended in phosphate buffer saline and measured for luminescence levels in a 96 well plate. Error bars represent standard deviation of 5 replicates, and the asterisk above represents a significant difference according to Mann-Whitney Rank Sum Test (p<0.01);

FIG. 2 is a graph showing the effect of Pseudomonas aeruginosa's total volatiles on a LuxR-expressing biosensor. Relative luminescence levels of E. coli/pSB401 reporter strain, expressing LuxR response regulator, in response to total volatiles of P. aeruginosa (PAO1) or 1 pmol of 3-oxo-C6-HSL (AHL) was measured as in FIG. 1. Antagonistic or synergistic effects of volatiles were examined with addition of 1 pmol of 3-oxo-C6-HSL to the reporter (PAO1+AHL). n=8; Error bars are the standard error of the mean. Different letters indicate a statistical difference (P<0.05) according to ANOVA on Ranks and Student-Newman-Keuls post hoc test;

FIGS. 3A-3B illustrate the volatiles profile of P. aeruginosa PAO1 wild type and a lasR-deficient mutant P. aeruginosa. FIG. 3A: Gas chromatogram of a wild type (PAO1—black line) and a lasR mutant (ΔlasR—red line) of P. aeruginosa. FIG. 3B: Abundance of five volatiles in the P. aeruginosa wild type and ΔlasR strains. n=3; Error bars represent standard error of the mean, asterisks represents significant difference between ΔlasR (grey) and PAO1 wild type (black) according to student t-test (P<0.05). Note the abundance of 2-AA in the wild-type P. aeruginosa volatiles. To aid visualization, results are presented only for retention times of 12-20 min. Beyond these retention times there were no substantial differences in volatile profiles of mutant compared to wild type strains;

FIG. 4 is a graph showing the effect of 2-AA and C6-HSL on E. coli/pSB401 luminescence. Relative luminescence of E. coli pSB401 reporter strain in the presence of 5 nM of C6-HSL (pSB401+C6HSL) or 50 μM of 2-AA (pSB401+2AA) was measured as in FIG. 1. Luminescence of the strain without any addition was measured as a control (pSB401). Measurements were carried out at 20 h post exposure to signaling molecules. Error bars represent standard deviation of four replicates, and the letters above represent the significant statistical groups ANOVA and Tukey Post-hoc (p<0.05);

FIGS. 5A and 5B are graphs illustrating the effect of 2-AA and 3-oxo-C6-HSL on LuxR-expressing biosensors. 2-AA (0-500 μM)(black columns) and 3-oxo-C6-HSL (0-500 nM)(red columns) were added to E. coli/pSB401 (5A) and E. coli JLD271/pAL103 (5B) reporter strains in order to evaluate the effect of 2-AA on LuxR response regulator. For antagonistic/synergistic assays (3-oxo-C6-HSL+2AA), 0-500 nM of 2-AA was added to the reporter strain in presence of 10 nM of 3-oxo-C6-HSL (green columns). Values represent the luminescence measurements taken 12 h post exposure to signaling molecules. n=4; Error bars represent standard error of mean, asterisks indicate a statistical difference (P<0.01) compared to control, according to ANOVA and Dunnett post hoc test;

FIG. 6 is a graph showing the effect of 2-AA on LuxR-regulated luminescence of Vibrio fischeri. Luminescence of wild type Vibrio fischeri MJ-1 in absence (Control, dark circle) and presence of 25 (open circle), 50 (dark triangle) and 100 μM (open triangle) of 2-AA and 10 nM of 3-oxo-C6-HSL (dark square) was measured every half hour during 18 hours incubation. Note the potent response to 100 μM 2-AA;

FIG. 7 is a graph showing the induction of luminescence in E. coli/pSB401 by 2-AA applied as a volatile. Relative luminescence of E. coli/pSB401 was measured after overnight exposure to 1 μg of 2-AA (1) added to the opposite compartment of a bipartite petri dish, compared with controls receiving none (0). Colonies of reporter strains were scraped from the agar, resuspended in PBS and measured in a 96 well plate. Error bars represent standard deviation of four replicates, and the asterisk represents significant difference between the groups according to t-test (P<0.05);

FIG. 8 is a graph showing the effect of 2-AA and C-12 HSL on P. aeruginosa LasR/pKD201 luminescence. Relative luminescence of P. aeruginosa JP2/pKD201 reporter strain was assayed in the presence of 50 μM of C12-HSL (JP2+C12HSL) or 2-AA (JP2+2AA). Luminescence of the strain without any addition was measured as a control (JP2). Measurements were carried out at 10 h post exposure to signaling molecules. Error bars represent standard deviation of five replicates, and the letters above represent the significant statistical groups, ANOVA and Tukey Post-hoc (p<0.05);

FIG. 9 is a graph showing the effect of 2AA and C4-HSL on P. aeruginosa's RhlR receptor. Relative luminescence of P. aeruginosa JP2/pKD-RhlA reporter strain, in presence of 50 μM of C4-HSL (RhlA+C4HSL) or 2-AA (RhlA+2AA). Luminescence of the strain without any addition was measured as a control (RhlA). Measurements were carried out at 20 h post exposure to signaling molecules. Error bars represent standard deviation of six replicates, and the letters above represent the significant statistical groups, ANOVA and Tukey Post-hoc (p<0.05);

FIG. 10 is a graph showing the effect of 2-AA and related modified molecules on a LuxR-expressing biosensor. Relative luminescence of E. coli pSB401 reporter strain was measured upon exposure to 50 μM of the following substances: 2-aminoacetophenone (2-AA), 3-aminoacetophenone (3-AA), 4-aminoacetophenone (4-AA), 2-nitroacetophenone, methyl anthranilate, anthranilic acid, acetophenone and 2-aminobenzaldehyde. Measurements are after 20 h of incubation. n=6; Error bar represent standard error of the mean. Different letters indicate a statistical difference (P<0.05) according to ANOVA and Student-Newman-Keuls post hoc test. Structure of each molecule is depicted above the corresponding bar;

FIGS. 11A and 11B illustrate the in silico docking of 3-oxo-C6-HSL (11A) and 2-AA (11B) into a LuxR molecular model. Interactions are shown as dotted lines and colored according to interaction type. Hydrogen bonds—green, hydrophobic interactions—pink;

FIG. 12 is a multiple amino acid sequence alignment of the LuxR response regulators (receptor proteins) of Vibrio fischeri (SEQ ID NO: 1), Aliivibrio logei (SEQ ID NO: 2), Vibrio mimicus (SEQ ID NO: 3), Photobacterium leiognathi (SEQ ID NO: 4) and Vibrio parahaemolyticus (SEQ ID NO: 5) (alignment according to T-Coffee algorithm). Residues that were shown to interact with 2-AA are marked in red;

FIGS. 13A-13B are graphs of the mass spectrometry analysis of 2-AA. FIG. 13A shows the Total Ion Chromatogram measurements (TIC); FIG. 123 shows the Selected Ion Monitoring spectrum (SIM) of the 100 ppm 2-AA standard sample;

FIGS. 14A-14C are graphs showing a mass spectrometry analysis of Pus sample number 1. FIG. 14A shows total Ion Chromatogram measurements (TIC); FIG. 14B shows the Selected Ion Monitoring spectrum (SIM) of the pus sample, and FIG. 14C shows the TIC overlay of 2-AA 1 ppm standard (blue) and the Pus sample number 1 (red) as in FIG. 14A;

FIGS. 15A-15B are graphs of the mass spectrometry analysis of Pus sample number 2. FIG. 15A shows the Total Ion Chromatogram measurements (TIC); FIG. 15B shows the Selected Ion Monitoring spectrum (SIM) of the pus sample;

FIGS. 16A-16B are graphs showing an analysis of 2-AA in pus samples using mass spectrometry and reporter strains. FIG. 16A shows an overlay of the TIC analysis presented in FIGS. 14 and 15: 1 ppm 2-AA standard (red), P. aeruginosa positive pus sample 1 (green), P. aeruginosa negative pus sample (black); FIG. 16B shows the relative luminescence of the E. coli pSB401 reporter strain upon exposure to volatiles emitted from pus sample 1 (positive for 2-AA in MS analysis and positive for P. aeruginosa in culture test) and pus sample 2 (negative for 2-AA in MS and negative for P. aeruginosa in culture test);

FIG. 17 is a graph showing the analysis of 2-AA in horse wound samples using reporter strains. The graph shows the relative luminescence of E. coli pSB401 reporter strain upon exposure to volatiles emitted from horse wound tissue sample number 1 (two days after treatment with antibiotics) and horse wound tissue sample number 2 (one week after treatment with antibiotics).

FIG. 18 is a schematic view showing a simplified form of the device of the invention, with a representative reporter cell immobilized on a solid support;

FIG. 19 is a schematic view showing a simplified form of the device of the invention, with a representative reporter cell encapsulated within a gas-permeable matrix on a solid support;

FIG. 20 is a schematic view showing a simplified form of the system of the invention, comprising the device and a sensor.

### DESCRIPTION OF SPECIFIC EMBODIMENTS OF THE INVENTION

The present invention, in some embodiments thereof, relates to a biosensor device comprising a reporter cell attached to or encapsulated within a solid support, the cell capable of producing a detectable signal upon contacting a luxR-like receptor ligand.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Effective and reliable detection and identification of bacteria is an ongoing challenge for medicine, environmental science and industry. Conventional methods commonly require time-consuming and costly culture of suspected bacterial-containing sample material for often complex analytical procedures.

Certain bacteria, such as Pseudomonas aeruginosa interact with their environment through secreted factors such as quorum sensing (QS) signals, activators of gene clusters modulating responses according to population density. The present inventors have shown that one such secretion in P. aeruginosa, the volatile organic compound (VOC) 2-aminoacetophenone (2-AA) 2-AA binds to and activates the LuxR-like receptor transcription factor.

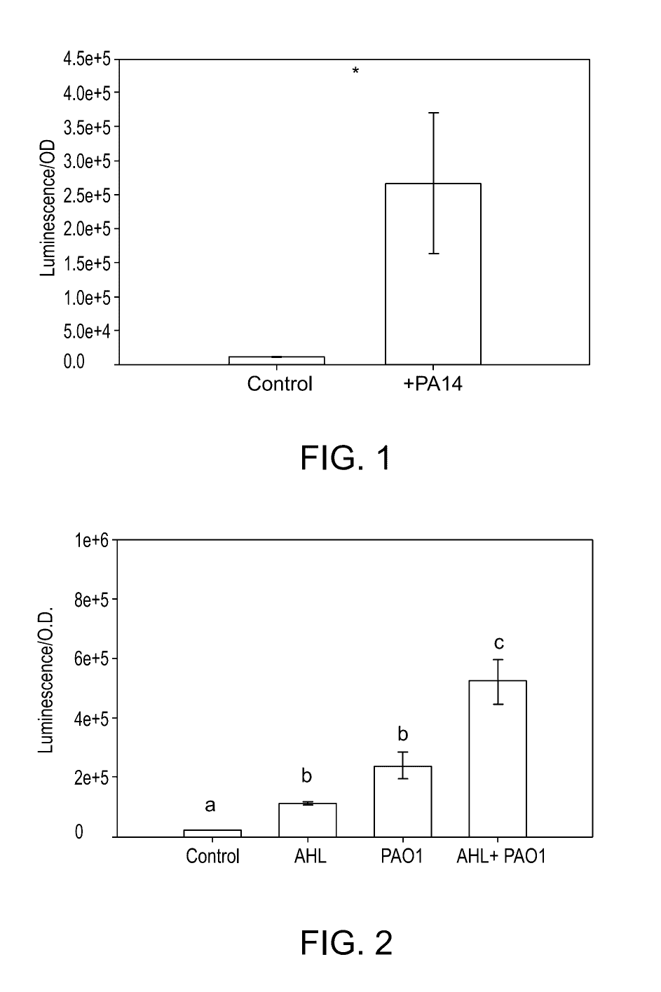

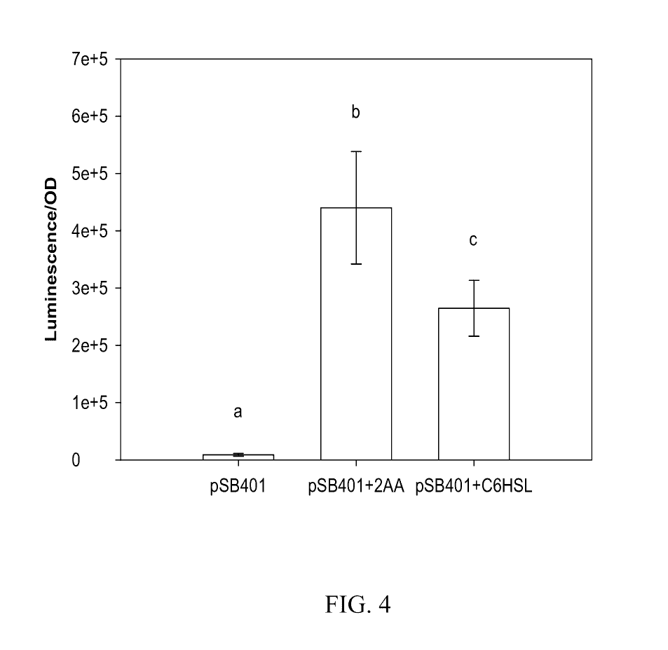

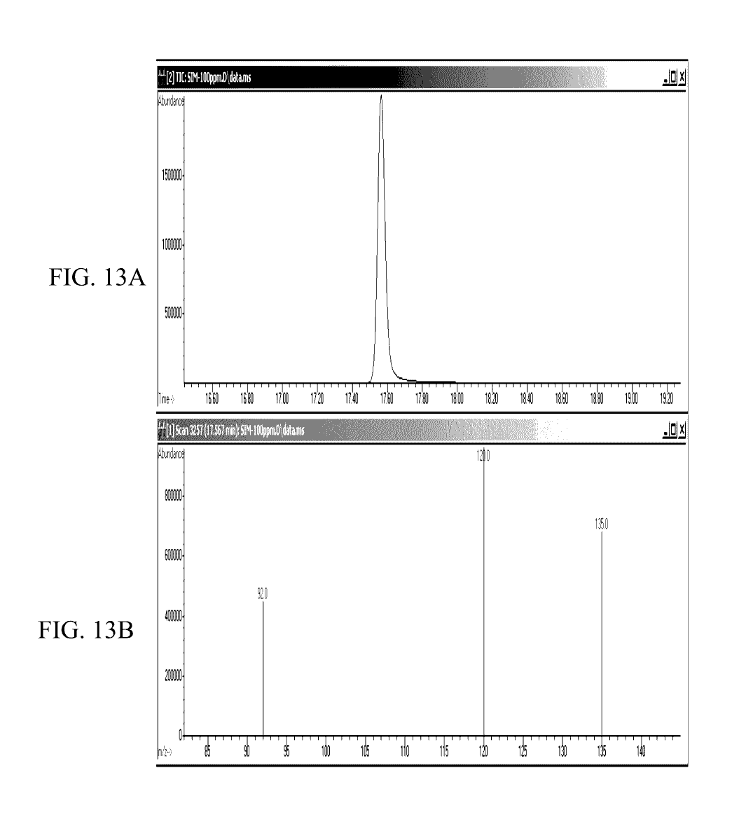

While reducing the present invention to practice, the present inventors have shown that a reporter cell comprising a LuxR-like receptor and a polynucleotide comprising a luxR-like receptor binding element transcriptionally fused to nucleic acid sequence encoding a reporter molecule can effectively detect 2-AA as both a pure compound (see FIGS. 4, 5A-5B, 6 and 7) and when secreted from P. aeruginosa (see FIGS. 1, 2 and 3A-3B). 2-AA detection by the biosensor reporter cell was as effective as gas chromatography in detecting P. aeruginosa in samples from suspected infected tissue (see FIGS. 13A-13B, 14A-14C, 15A-15B, 16A-16B and 17). Devices comprising such biosensor reporter cells, encapsulated and/or attached to a solid support can be effectively employed for 2-AA and bacterial detection in a wide variety of medical, scientific, environmental and industrial applications.

Thus, according to one aspect of the present invention there is provided a device comprising:

A reporter cell comprising a polynucleotide comprising:

a) a first nucleic acid sequence encoding a reporter molecule capable of producing a detectable signal, and

b) a second nucleic acid sequence comprising a luxR-like receptor binding element for regulating transcription of the sequence encoding the reporter molecule;

wherein the reporter cell is attached to a solid support or encapsulated within an encapsulation matrix attached to the solid support.

As used herein, the phrase “reporter cell” refers to a cell which can be genetically engineered i.e., transformed or infected with an exogenous polynucleotide.

As used herein, the term “exogenous” or “foreign” refers to genetic material (e.g. DNA, RNA) which is not normally a component of the genetic material of the wild-type, or non-engineered, untransformed organism.

The reporter cell of the present invention can be a bacterial reporter cell, a fungal reporter cell, a plant reporter cell, an algal reporter cell, a protozoan reporter cell and an animal reporter cell.

In some embodiments, the reporter cell is a bacterial reporter cell. The bacterial reporter cell can be a Gram-positive or a Gram negative bacterial cell. Exemplary bacterial reporter cells suitable for the present invention include, but are not limited to Escherichia, Pseudomonas, Vibrio, Bacillus, Staphylococcus, Alcaligenes, Acinetobacter, Synechococcus, Aeromanas hydrophilia and Ralstonia. In some embodiments the reporter cell is V. fischeri MJ-1, which has native luxR receptor, reporter and binding elements. In some embodiments the reporter cell is a genetically modified E. coli, including, but not limited to an E. coli harboring a pSB401 plasmid expressing LuxR of V. fischeri and E. coli JLD271/pAL103 harboring a pAL103 plasmid expressing LuxR of V. fischeri.

In some embodiments of the present invention the reporter cell is a bacterium that contains a polynucleotide comprising the luxR-like receptor binding element and the complete luxCDABE gene cassette from V. fischeri and that is specific for the luxR receptor ligands. Any suitable bacteria can be used as the reporter cells, but in some embodiments, the bacteria used should be devoid of expression of the luxI autoinducer (AI), in order to avoid autoinduced feedback loops upon binding of the LuxR-like receptor to its ligand and activation of the luxR cassette. Within the reporter cells, expression of endogenous Lux-R-like signaling system components can be a potential confounding factor, interfering with the accurate detection and reporting of events such as binding of a ligand (e.g. 2-AA) to the LuxR-like receptor protein, or binding of the LuxR-like receptor-ligand complex with the receptor binding element. Thus, in some embodiments, the host cell is devoid of endogenous luxR-like expression. In other embodiments, the host reporter cell is devoid of endogenous luxR-like agonists or antagonists or both.

Thus, in some embodiments of the present invention the reporter cell is devoid of endogenous luxR-like receptor expression. In other embodiments, the reporter cell is devoid of endogenous luxR-like agonists or antagonists or both. In specific embodiments, the reporter cell is devoid of luxI expression. In order to determine whether candidate cells are indeed devoid of endogenous luxR-like receptor expression, the presence of DNA sequences encoding a luxR-like receptor, or RNA transcripts of such sequences can be detected by PCR, the actual luxR-like receptor protein can be detected, for example, immunologically, and binding of luxR-like ligands can be assessed both with the whole cell or in a cell free fraction thereof. Similar assays can be employed to detect endogenous luxR-like agonists or antagonists, or luxI. Expression of luxR-like agonists and antagonists, and luxI, can also be evaluated by observing the effect of candidate cells, cell extracts or fractions thereof on binding to isolated luxR-like receptors, or on the function of luxR-like receptor signaling in cell free or whole cell preparations. In some embodiments of the invention, the reporter cell of the invention is a naturally occurring cell, which is naturally capable of binding a ligand of the luxR-like receptor. Typically, however the “reporter cell” is a genetically engineered cell, harboring a luxR-like receptor, which is foreign to the cell.

In other embodiments of the invention, the reporter cell comprises a third nucleic acid sequence encoding a luxR-like receptor capable of binding 2AA.

The term “lux-R-like receptor” or “response regulator” relates to a polypeptide receptor comprising a ligand binding domain (e.g. 2-AA binding domain) and a DNA binding domain which recognizes a luxR-like receptor binding nucleotide sequence. Typically, luxR-like receptors bind acyl-homoserine lactones with six or eight carbons in their side chains such as 2-AA. These carbon chains can be either oxygenated or not oxygenated, referring to: N-3-(oxohexanoyl) homoserine lactone, N-3-(oxooctanoyl) homoserine lactone, N-hexanoyl homoserine lactone, N-octanoyl homoserine lactone. Bacterial species harboring the LuxR-like receptors include, but not restricted to, Vibrio fisscheri, Pectobacterium carotovorum, Burkholderia cepacia, Serratia liquefaciens, Serratia plymuthica, Pseuodomonas aureofacians, Pseudomonas syringae, Chromobacterium violaceum and Halomonas anticariensis.

In some embodiments, the term “luxR-like receptor” refers to a cognate cytoplasmic transcription factor of a LuxI/LuxR-type quorum sensing (QS) system homologous to the QS system from bioluminescent marine bacterium Vibrio fischeri (SEQ ID NO: 6). In LuxI/LuxR systems, the physiological LuxI homolog is an autoinducer (AI) synthase that catalyzes a reaction between SAM and an acyl carrier protein (ACP) to produce a freely diffusible acyl homoserine lactone (AHL or HSL) autoinducer (AI). At high concentrations, AHL AIs bind to the cytoplasmic LuxR-like transcription factors (LuxR-like receptors), which, when not bound by AI, are rapidly degraded. AI binding stabilizes the LuxR-like proteins, allowing them to fold, bind to a luxR-like receptor binding element in the DNA, and activate transcription of target genes. Typically, AHL (HSL)-bound LuxR-like receptor proteins also activate luxI expression, forming a feed-forward autoinduction loop that floods the vicinity with AI.

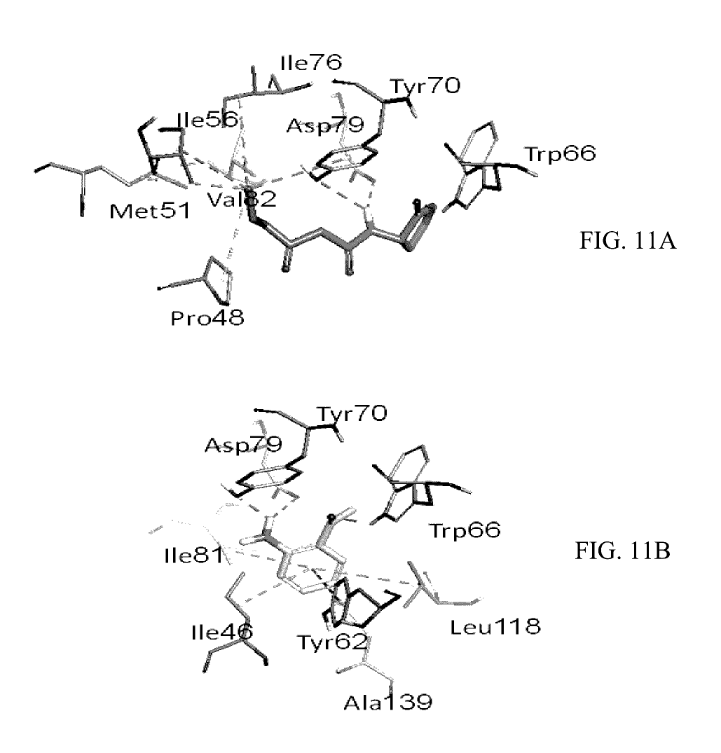

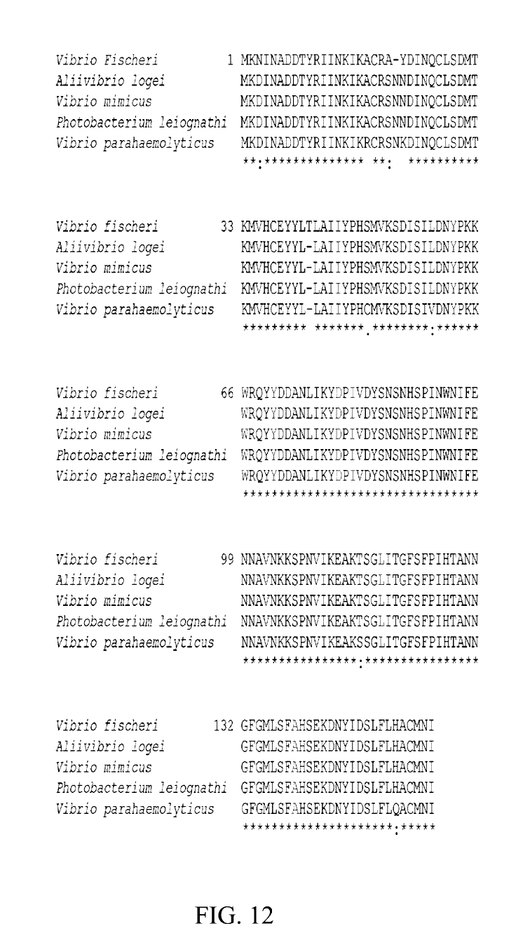

LuxI/LuxR homologs have been identified in more than 100 Gram-negative bacterial species (see, for example, Case et al, ISME J, 2008; 2:345-49). Using in-silico docking techniques, the present inventors have defined a potential docking site for 2-AA in the V. fischeri LuxR-like receptor (SEQ ID NO: 6), and identified a number of amino acid residues critical to proper fit of the ligand within the binding pocket (see FIGS. 11A and 11B and 12). Reporter cells not responsive to 2-AA or P. aeruginosa were found to harbor QS signaling receptor proteins which lacked the putative critical residues. Thus, in some embodiments of the present invention, the LuxR-like receptor protein comprises a LuxR-like homologue or ortholog having conserved amino acid residues corresponding to amino acids Trp66, Try70 and Asp79, and to Tyr62, Leu118, Ala139, Ile46 and Ile 81 of the Vibrio fischeri LuxR-like receptor protein (SEQ ID NO: 6). Exemplary LuxR-like receptors suitable for use in some embodiments of the present invention and having highly similar sequences including putative 2-AA-binding residues include, but are not limited to the LuxR-like receptors of Vibrio fischeri (SEQ ID NO: 6), Aliivibrio logei (SEQ ID NO: 7), Vibrio mimicus (SEQ ID NO: 8), Photobacterium leiognathi (SEQ ID NO: 9) and Vibrio parahemolyticus (SEQ ID NO: 10). Exemplary nucleic acid sequences encoding LuxR-receptor proteins include SEQ ID NOs: 11, 12, 13, 14 and 15).

In some embodiments of the invention, the LuxR-like receptor protein comprises the Vibrio fischeri LuxR-like receptor protein (SEQ ID NO: 6). In some embodiments the LuxR-like receptor protein is encoded by the nucleic acid sequence SEQ ID NO: 11.

The reporter cell comprises a first nucleic acid sequence encoding a reporter molecule capable of producing a detectable signal translationally fused to the second nucleotide sequence comprising the luxR-like receptor binding element. The luxR-like receptor, which is a transcription factor, activates the transcription of genes via the receptor binding element once it binds to the signaling molecule (e.g. 2-AA).

As used herein, the term “reporter molecule” refers to a gene transcript, the transcription of which results in production of a detectable signal. The detectable signal, as used herein, can be any change in the reporter cell or its environment, which can be then detected, resulting from the transcription of the reporter molecule.

In some embodiments, the reporter molecule is an mRNA for a polypeptide (e.g. enzyme) which catalyzes the production of a detectable signal (e.g. luciferase). In some embodiments, the reporter molecule is a polynucleotide regulatory factor, or encodes a peptide or polypeptide regulatory factor which enables (e.g. induces) production of a detectable signal by other polypeptides. Regulatory factors, such as transcription enhancers and enzyme inducers can act at multiple levels to effect the production of the detectable signal.

In some embodiments of the invention, the nucleic acid sequence encoding a reporter molecule is a lux luciferase gene cluster or “cassette”. As used herein, “gene cluster” refers to a plurality of gene sequences which encode gene products which are components of the same biochemical pathway, e.g luciferase bioluminescence production. The cluster of activated genes luxCDABE are involved in synthesis and activation of the luciferase gene, which emits light once activated (luminescence).

As used herein, the term “cassette” refers to a recombinant DNA construct made from a vector and inserted DNA sequences. The complete lux cassette comprises five genes, i.e. luxA, B, C, D and E. LuxA and luxB that encode the proteins that are responsible for generating bioluminescence while luxC and D encode an aldehyde required for the bioluminescence reaction. In a specific embodiment the lux cluster is the luxCDABE cluster of V. fischeri (SEQ ID NO: 16).

Although the experiments described herein involve the use of luxCDABE from V. fischeri (SEQ ID NO: 16), the lux cluster or cassette can be from other luminescence-producing bacteria including Photorhabdus luminescens or Vibrio harveyi. In addition, the reporter molecule can be insect luciferase (luc from the firefly or click-beetle).

Besides luminescence, reporter cells can also be made to generate signals that are fluorescent (using green fluorescent protein, SEQ ID NO: 17, or red fluorescent protein, SEQ ID NO: 18, orange fluorescent protein SEQ ID NO: 19) or derivatives that fluoresce in cyan, red, or yellow wavelengths as well as aequorin or uroporphyrinogen III methyltransferase (UMT)). Colorimetric (lacZ, xylE, bla), chemiluminescent, and electrochemical signals can also be implemented within the invention. Non-limiting examples of molecules producing detectable signals moieties suitable for the present invention are provided in Table 1.

TABLE 1

| | Amino Acid sequence | Nucleic Acid sequence |
|---|---|---|
| | (GenBank Accession No.)/ | (GenBank Accession No.)/ |
| Identifiable Moiety | SEQ ID NO: | SEQ ID NO: |
| Green Fluorescent protein | AAL33912/17 | AF435427/27 |
| Alkaline phosphatase | AAK73766/20 | AY042185/30 |
| Peroxidase | CAA00083/21 | A00740/31 |
| Histidine tag | Amino acids 264-269 of | Nucleotides 790-807 of |
| | GenBank Accession No. | GenBank Accession No. |
| | AAK09208/22 | AF329457/32 |
| Myc tag | Amino acids 273-283 of | Nucleotides 817-849 of |
| | GenBank Accession No. | GenBank Accession No. |
| | AAK09208/23 | AF329457/33 |
| Biotin lygase tag | LHHILDAQKMVWNHR/24 | |
| orange fluorescent protein | AAL33917/19 | AF435432/29 |
| Beta galactosidase | ACH42114/25 | EU626139/34 |
| Streptavidin | AAM49066/26 | AF283893/35 |
| Amcyan | AEI59072.1/44 | JF796087.1/45 |

As used herein, the phrase “LuxR-like receptor binding element” refers to a DNA sequence which can bind a LuxR-like receptor, following binding of a LuxR-like receptor ligand to the LuxR-receptor and which can activate transcription of a nucleic acid sequence in a cis manner. It will be appreciated that the nucleic acid sequence comprising a luxR-like receptor binding element of the reporter cell of the invention is for regulating transcription of a first nucleic acid sequence encoding a reporter molecule capable of producing a detectable signal.

In Vibrio fischeri the native LuxR receptor-ligand complex (LuxR/3-oxo-C6-HSL) binds to a 20-bp luxR-receptor binding sequence within the luxR-luxI intergenic region referred to as the ‘lux box’. The lux box is centered 42.5 bp upstream of the luxI promoter start site, indicating the LuxR/3-oxo-C6-HSL complex serves as a transcriptional activator. In the V. fischeri LuxR QS system, lux box base pairs located at positions 3-5 and 16-18 are critical for LuxR regulation of lux expression. Binding of the ligand in the LuxR/3-oxo-C6-HSL complex renders the LuxR receptor protein resistant to proteolysis.

LuxR-like receptor binding elements are also found outside of the lux locus in V. fischeri: greater than 20 genes have been shown to be significantly differentially regulated in the presence of physiological concentrations of 3-oxo-C6 (Antunes et al, J Bacteriol 2007; 189:8387-91). The LuxR/3-oxo-C6 complex has been shown to directly bind to 7 of the corresponding promoter elements in these genes.

Thus, in some embodiments of the present invention, the luxR-like binding element of the second nucleic acid sequence comprises a V. fischeri lux box sequence (SEQ ID NO: 36). Lux-box homologues, which can bind some luxR-like receptor proteins include but are not limited to the tra box of Agrobacter tumefaciens (SEQ ID NO: 37), rhl box of Pseudomonas aeruinosa (SEQ ID NO: 38), Qsc102 (activated by lasR of Pseudomonas aeruginosa)(SEQ ID NO: 39), Qsc117 (SEQ ID NO: 40), phzA (SEQ ID NO: 41), cep box of Burkholderia cenocepacia (SEQ ID NO: 42) sequence and the las box of Pseudomonas aeruginosa (SEQ ID NO: 46). In some embodiments the luxR-like binding element of the second nucleic acid sequence comprises a V. fischeri lux box (SEQ ID NO: 36) sequence.

It will be appreciated that other bacterial cells which are responsive to 2-AA signaling may comprise genes regulated by additional luxR-like binding elements that can be suitable for use in the instant invention. Identification and characterization of such additional luxR-like binding elements can be performed by screening for differential gene expression in the presence and absence of the 2-AA ligand, detecting sequences homologous to known luxR-like binding elements and/or performing direct binding studies with candidate DNA sequences and luxR-ligand complexes.

In some embodiments, the reporter cell comprises a polynucleotide comprising the first nucleic acid sequence, the second nucleic acid sequence and the third nucleic acid sequence. In other embodiments, the third nucleic acid sequence is comprised on a polynucleotide distinct of said polynucleotide.

Nucleic acid sequences comprising the first nucleic acid sequence encoding a reporter molecule capable of producing a detectable signal, and second nucleic acid sequence comprising a luxR-like receptor binding element for regulating transcription of the sequence encoding the reporter molecule and optionally the third nucleic acid sequence encoding a luxR-like receptor of some embodiments of the invention may be optimized for expression for a particular host reporter cell type. Examples of such sequence modifications include, but are not limited to, an altered G/C content to more closely approach that typically found in the host cell species of interest, and the removal of codons atypically found in the host cell species commonly referred to as codon optimization.

The phrase “codon optimization” refers to the selection of appropriate DNA nucleotides for use within a structural gene or fragment thereof that approaches codon usage within the host cell of interest. Therefore, an optimized gene or nucleic acid sequence refers to a gene in which the nucleotide sequence of a native or naturally occurring gene has been modified in order to utilize statistically-preferred or statistically-favored codons within the host cell. The nucleotide sequence typically is examined at the DNA level and the coding region optimized for expression in the host cell species determined using any suitable procedure, for example as described in Sardana et al. (1996, Plant Cell Reports 15:677-681). In this method, the standard deviation of codon usage, a measure of codon usage bias, may be calculated by first finding the squared proportional deviation of usage of each codon of the native gene relative to that of highly expressed genes, followed by a calculation of the average squared deviation. The formula used is: 1 SDCU=n=1 N [(Xn−Yn)/Yn]2/N, where Xn refers to the frequency of usage of codon n in highly expressed genes, where Yn to the frequency of usage of codon n in the gene of interest and N refers to the total number of codons in the gene of interest.

One method of optimizing the nucleic acid sequence in accordance with the preferred codon usage for a particular cell type is based on the direct use, without performing any extra statistical calculations, of codon optimization tables such as those provided on-line at the Codon Usage Database through the NIAS (National Institute of Agrobiological Sciences) DNA bank in Japan (www.dotkazusadotordotjp/codon/). The Codon Usage Database contains codon usage tables for a number of different species, with each codon usage table having been statistically determined based on the data present in Genbank.

By using the referenced tables to determine the most preferred or most favored codons for each amino acid in a particular species (for example, E. coli), a naturally-occurring nucleotide sequence encoding a protein of interest can be codon optimized for that particular species. This is affected by replacing codons that may have a low statistical incidence in the particular species genome with corresponding codons, in regard to an amino acid, that are statistically more favored. However, one or more less-favored codons may be selected to delete existing restriction sites, to create new ones at potentially useful junctions (5′ and 3′ ends to add signal peptide or termination cassettes, internal sites that might be used to cut and splice segments together to produce a correct full-length sequence), or to eliminate nucleotide sequences that may negatively effect mRNA stability or expression.

A naturally-occurring encoding nucleotide sequence may already, in advance of any modification, contain a number of codons that correspond to a statistically-favored codon in a particular species. Therefore, codon optimization of the native nucleotide sequence may comprise determining which codons, within the native nucleotide sequence, are not statistically-favored with regards to a particular cell, and modifying these codons in accordance with a codon usage table of the particular species to produce a codon optimized derivative. A modified nucleotide sequence may be fully or partially optimized for host cell codon usage provided that the protein encoded by the modified nucleotide sequence is produced at a level higher than the protein encoded by the corresponding naturally occurring or native gene. Construction of synthetic genes by altering the codon usage is described in for example PCT Patent Application No. 93/07278.

To produce the reporter cell of the present invention using recombinant technology, polynucleotides comprising the first, second and optionally third nucleic acid sequence may be ligated into a nucleic acid expression vector (e.g. bacterial plasmid) or nucleic acid construct system, under the transcriptional control of a luxR-receptor binding element and cis-regulatory sequence (e.g., promoter sequence) suitable for directing transcription of the reporter molecule in the host cell. In specific embodiments, the lux box element and promoter comprise the nucleic acid sequence as set forth in SEQ ID NO: 43.

In some embodiments, polynucleotides comprising the first, second and optionally third nucleotide sequences are ligated into nucleic acid expression vectors for transformation and expression in the host cell. In certain embodiments, the first nucleic acid sequence encoding a reporter molecule for producing a detectable signal, second nucleic acid sequence comprising a luxR-receptor binding element and the third nucleic acid sequence encoding a luxR-like receptor protein are located on the same nucleic acid expression vector, and are thus located on the same polynucleotide within the host cell. Thus, in some embodiments, the reporter cell comprises a polynucleotide which comprises the first nucleic acid sequence encoding the reporter molecule, a second nucleic acid sequence comprising the luxR-like receptor binding element and the third nucleic acid sequence encoding the luxR-like receptor protein. In other embodiments, the third nucleic acid sequence is comprised on a polynucleotide distinct and separate from the polynucleotide comprising the first and second nucleic acid sequences, also referred to herein as a nucleic acid construct system.

In some embodiments of the invention, the vector is a bacterial plasmid, constructed using a commercially available bacterial plasmid “backbone”. In specific embodiments, the bacterial plasmid backbone used is pACYC184 or pBR322.

Thus, the present invention contemplates isolated polynucleotides comprising the first, second and optionally third nucleic acid sequences of the present invention.

The phrase “an isolated polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exon sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

In some embodiments of the present invention, the reporter cell is selected from the group consisting of a bacterial cell, a fungal cell, a plant cell, an algal cell and an animal cell comprising a nucleic acid sequence encoding a reporter molecule capable of producing a detectable signal (e.g. the luxR gene cluster). Eukaryotic host cells (yeast: Gupta et al, FEMS Yeast Res 2003 4:305; Sanseverino et al, Appl Environ. Microbiol 2005; 71:4455-60; and Human cells: Patterson et al, J Ind Microb Biotech, 2005; 3:2115-23 and Close et al, PLoS One, 2010; 5:e12441), as well as bacterial host cells have been engineered with luxR sequences to produce bioluminescent reporter cells. Thus, the expression vector or nucleic acid construct of the present invention can include additional sequences which render the vector suitable for replication and integration in prokaryotes, eukaryotes, or preferably both (e.g., shuttle vectors). Typical cloning vectors contain transcription and translation initiation sequences (e.g., promoters, enhancers) and transcription and translation terminators (e.g., polyadenylation signals).

Various methods can be used to introduce the expression vector of the present invention into the host cell system. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Exemplary bacterial based expression systems are disclosed in Baneyx et al., Current Opinion in Biotechnology, 1999; 10, 411-421 and Macrides et al, Microbiol Rev 1996, 60: 512-538, incorporated herein by reference.

The host cells may be transformed stably or transiently with the nucleic acid constructs of the present invention. In stable transformation, the nucleic acid molecule of the present invention is integrated into the host cell genome and as such it represents a stable and inherited trait. In transient transformation, the recombinant nucleic acid molecule is expressed by the transformed cell but is not integrated into the genome and as such represents a transient trait.

As mentioned, the reporter cell is attached to a solid support.

As used herein, the terms “attach,” “attachment,” “adhere,” “adhered,” “adherent,” “immobilize”, or like terms generally refer to immobilizing or fixing, for example, the reporter cell of the present invention, to a surface, such as by physical absorption, chemical bonding, and like processes, or combinations thereof. Particularly, “cell attachment,” “cell adhesion,” or like terms refer to the interacting or binding of cells to a surface, such as by culturing, or interacting with cells with a surface, such as a the surface of the solid support.

The solid support surface can be unmodified or modified, such as having a surface coating, an anchoring material, a compatibilizer (e.g., fibronectin, collagen, lamin, gelatin, polylysine, etc.), or like modifications that promote reporter cell adhesion and cell status or growth. For suspension cells, the cells can be, for example, brought into contact with the surface of the solid support through physical settlement during incubation, or through surface-cell interactions. The surface-cell interactions can be achieved by several means, e.g., covalently coupling of reactive surfaces with the cell surface (cell membrane, cell wall) proteins or molecules, charge-based electrical interactions, binding of the solid support surface molecules (e.g., antibody, ligand) with cell surface molecules, or like approaches.

Solid supports for attachment or immobilization of reporter cells are well known in the field, for example, US2012/0045835 to Michelini et al. Briefly, the solid support can be a natural polymer, such as a combination of collagen and/or its derivatives and proteoglycan, or a mixture of proteoglycans, wherein the source of the collagen (e.g., bovine, horse, pig, shark) is selected compatible with the biosensor vitality. The solid support can also be a synthetic polymer, for example, a combination of a synthetic vinyl polymer with an optionally modified polysiloxane, ensuring both structural rigidity and reporter cell confinement and sufficient transparency for optical signal transmission to the detector, where the detectable signal is an optical signal (e.g. bioluminescence). Suitable polymers include, but are not limited to the vinyl polymer polyvinylpyrrolidone (PVP), collagen-proteoglycan mixtures, and the like.

The solid support can also be a natural material such as paper, wood, metal, natural polymers, or a synthetic material, or a mixture of natural and synthetic materials, such as mixed natural or synthetic polymers, depending on the cell type being immobilized.

Further, synthetic polymers, can comprise or be added to the immobilization/attachment mixture.

In another embodiment of the present invention, a vegetable mucilage is added to increase adhesiveness of the cells to the solid support. A small percentage is enough. Exemplary vegetable mucilages are commercially available from mauve and aloe. Solid supports for cell attachment should also be prepared with an appropriate buffered solution according to the type of reporter cell.

The immobilization of cells to the solid support is effected without impairing the cellular integrity, vitality and functionality (i.e., ability to respond to 2AA) of the reporter cell. In one exemplary method of immobilization, the cells are cultured to desired density, optionally rinsed free of medium, and mixed with a coupling agent such as glutaraldehyde, hexamethylene diisocyanate and hexamethylene diisothiocyanate, before being applied to the solid support surface. As mentioned, immobilization to the solid support may include immobilization using polyacrylamide, immobilization using natural polymers such as alginic acid, collagen, gelatin, agar and kappa-carrageenan, and immobilization using synthetic polymers such as photosetting resins and urethane polymers.

One advantage of biosensors such as the reporter cell described herein is their sensitivity, small size and simplicity of operation. The reporter cells can be easily contacted with test samples or even deployed for detection of luxR-like ligands (e.g. 2-AA) or microorganisms producing them in situ, for example, at the site of a wound (e.g. a burn, infection, inflammation) or suspected contaminated surface or material (e.g. medical device, water source, breath or air).

Immobilization of the reporter cell on the solid support of the device allows locating of the device comprising the immobilized reporter cells in proximity with the source of suspected LuxR-like receptor ligand (e.g. 2-AA), simplifying detection and potentially even eliminating the need for sample retrieval. Thus, in one embodiment of the invention, the device configured such that it allows positioning of the reporter cells in proximity with the source of suspected LuxR-like receptor ligand (e.g. 2-AA).

Thus, according to some embodiments of the present invention, there is provided a device comprising a reporter cell as described herein attached to a solid support, or encapsulated within a gas permeable encapsulation matrix.

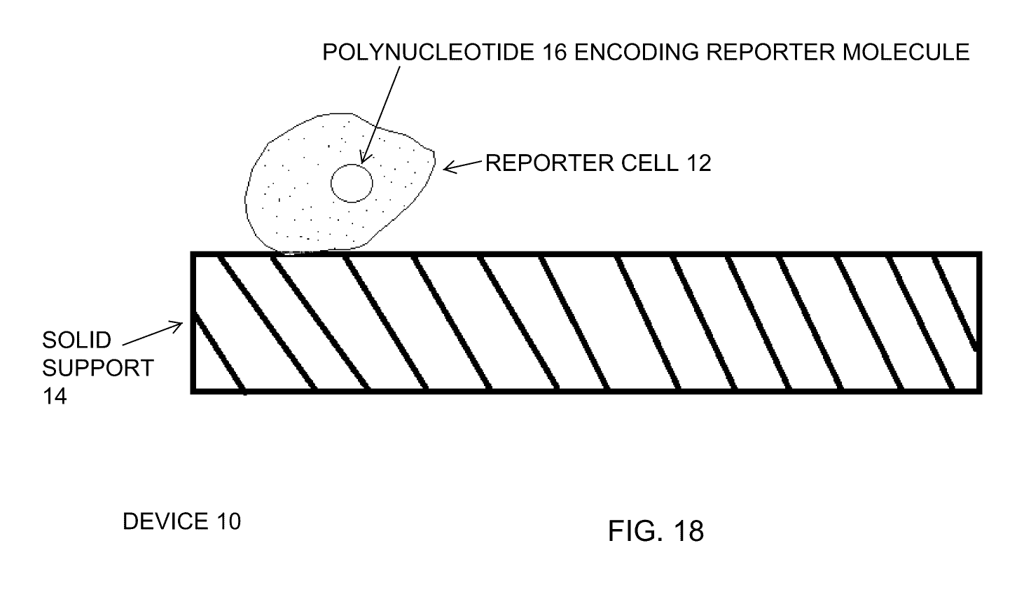

FIG. 18 illustrates an exemplary device 10 of one embodiment of the present invention, having reporter cell 12, which comprises the polynucleotide 16 having the first and second nucleotide sequences as described herein, the reporter cell immobilized on the solid support 14.

Solid support 14 can be any rigid or semi-rigid material to which cells, such as bacterial or yeast cells can be attached, of the cells.

In some embodiments of the present invention, the reporter cell 12 is attached to the solid support 14. In other embodiments, reporter cell 12 is encapsulated within an encapsulation matrix.

Suitable encapsulation matrices, which maintain cell viability, allow for detection of the detectable signal and provide gas permeability (e.g. for cell contact with volatile luxR-like receptor ligands such as 2-AA) are known in the field. For example, Smith et al (US 2008/0182287) teaches cell encapsulation matrices from PBP block polymers characterized by low toxicity and optical compatibility for the manipulation, analysis or processing of live cells. PBP gel properties can be modified by formulation providing block polymers with different transition temperatures suitable for different applications.

In another embodiment, polymers, such as polyvinylpyrrolidone (PVP), and polysiloxanes, optionally modified and/or crosslinked with an orthosilicate, can be used at different concentrations, typically from 0.05 to 15%, to create a matrix suitable to encapsulate cells and maintain their vitality, meanwhile assuring transparency, which is a crucial factor for the detection of luminescent signals. A modified polysiloxane is a polysiloxane with alkyl, acrylate, alcohol groups. Polysiloxanes are polymers with a main backbone Si—O—Si of 30-60 Si atoms length and lateral chains from C₁ to C₁₂.

Orthosilicate is a suitable crosslinking agent, and in some embodiments the polysiloxane is dimethylsiloxane, crosslinked with tetraethyl-orthosilicate.

In other embodiments, the encapsulation matrix can be an agar matrix, or any other suitable solid or semi-solid culture medium, alone or covered (e.g. coated) with a gas-permeable retaining layer. Also, alginate has been successfully used for encapsulation of cells without adverse effects on viability. Long-term viability (weeks to months) is possible as long as the alginate-encased cells remain moist. Latex copolymers have also been reported to be useful for immobilizing E. coli and maintaining viability. Other matrices include carrageenan, acrylic vinyl acetate copolymer, polyvinyl chloride polymer, sol-gel, agar, agarose, micromachined nanoporous membranes, polydimethylsiloxane (PDMS), polyacrylamide, polyurethane/polycarbomyl sulfonate, or polyvinyl alcohol. Electrophoretic deposition may also be employed.

Reporter cells can be encapsulated, for example, by mixing with the encapsulation matrix in a sol state, prior to gelling, and forming the mixture into the desired shape (beads, blocks, particles, etc) until gelled, suitable for attachment to the solid support.

It will be appreciated that the encapsulation matrix can be part of, or incorporated into the solid support, so that the reporter cells are distributed within the body of the solid support. In such an embodiment, the solid support can advantageously have some of the characteristics of the encapsulation matrix—for example, minimal or no interference with the detectable signal (e.g. optical transparency), maintenance of reporter cell viability and function and gas permeability.

It will be appreciated that signal detection in the device of the present invention can be enhanced by attachment of large numbers of reporter cells to the solid support, or by inclusion of large numbers of reporter cells in the encapsulation matrix. Thus, in some embodiments, the device comprises a population of reporter cells. Limitations to the number of cells comprised in the device are determined by the type of cell, size of the solid support or encapsulation matrix, sensitivity of the sensor for detecting the detectable signal, intended use (e.g. expected abundance of LuxR-1 ligand, physical constraints of the test environment, etc). Suitable reporter cell population sizes for effective use in the device of the present invention can be determined by monitoring the detectable signal (e.g. bioluminescence) in serial dilutions of reporter cell cultures following exposure to the LuxR-like ligand (e.g. 2-AA) or organism producing the LuxR-like ligand. Calibration with amounts or concentrations of the ligand or of the organisms producing the ligand within the range expected to be present in the samples, surfaces or objects to be tested can further aid accurate determination of the numbers of reporter cells needed for the device.

In some embodiments of the invention, the reporter cells are contained on, or contacted with a nutrient medium, such as nutrient agar. The nutrient medium can be added on to the solid support, or can be integrated within the solid support. In some embodiments, the reporter cell or cells are disposed upon a nutrient medium in a culture vessel, such as a culture dish, a flask or a multi-well culture plate.

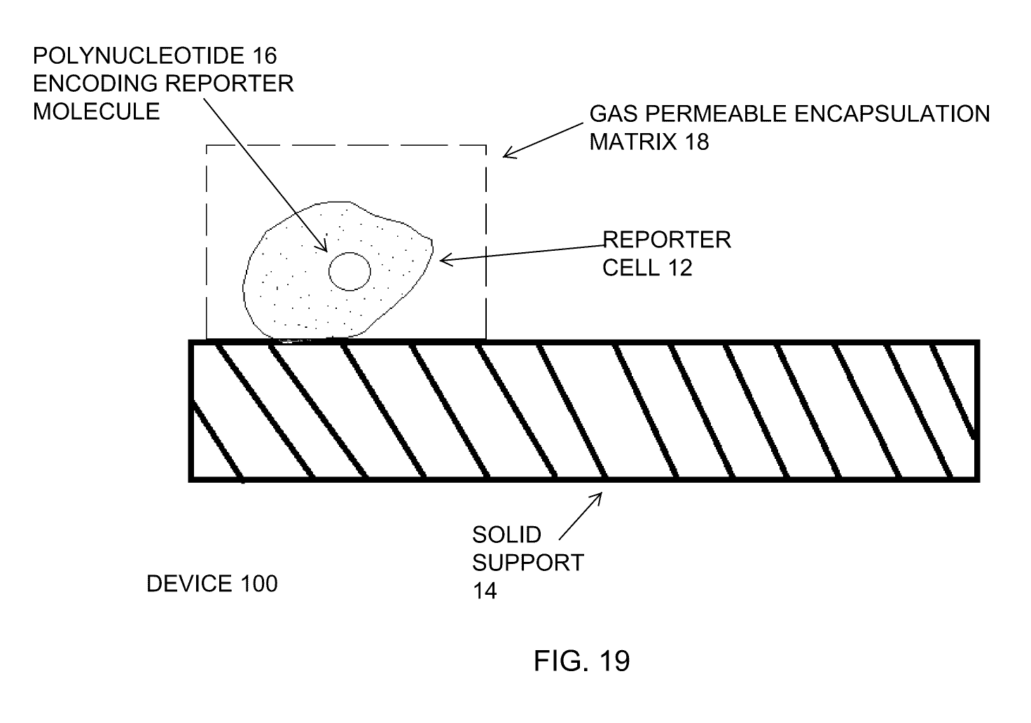

FIG. 19 illustrates an exemplary device 100 of one embodiment of the present invention, having reporter cell 12, which comprises the polynucleotide 16 having the first and second nucleotide sequences as described herein, the reporter cell encapsulated within encapsulation matrix 18, which is immobilized on the solid support 14.

In some embodiments of the present invention, the device is comprised in a system which further comprises a sensor.

As used herein, the term “sensor” refers to a detector device capable of detecting the detectable signal produced by the reporter cell. In some embodiments, where the nucleic acid sequence encoding the detectable signal is luminescence or bioluminescence, and the reporter molecule capable of producing a detectable signal can be a protein produced upon luxR-like activation that is capable of giving luminescence such as luciferase, the detectable signal can read by a luminometer or the luminescence can be read by using photodiodes and a signal-processing system to translate the reporter assay from a luminescent signal to an electric signal. Some methods for detection of luminescence are described in (Vijayaraghavan et al, 2007) and (Li et al, 2012).

Systems which comprise the device of the present invention may comprise a variety of different sensor modalities at essentially any location on the device. Detection can be achieved using sensors that are incorporated into the device or that are separate from the device but aligned with the region of the device to be detected.

A sensor typically comprises a signal receiver (detector) and a transducer for converting the detected signal into an energy form that can easily be transmitted, quantified and stored. The type of sensor is determined, of course, by the nature of the detectable signal. Thus, in some embodiments, the sensor is selected from the group consisting of an optical sensor, an electrochemical sensor and a chemical sensor. In some embodiments the device can also comprise a signal amplifier for increasing the sensitivity level of detection of the signal.

In other embodiments, wherein the detectable signal is luminescence or fluorescence, the light response generated by luminescent reporter cells, whether bacterial, yeast or otherwise, is typically measured with optical transducers such as photomultiplier tubes, photodiodes, microchannel plates, or charge-coupled devices. Some means of transferring the bioluminescent signal to the transducer is additionally required, which, in large units necessitates the need for fiber optic cables, lenses, or liquid light guides. However, hand-held, battery operated photomultiplier units that can interface with a laptop computer or wireless devices with memory and computing capacity, such as “smartphones” are available (The Azur Corporation, Carlsbad, Calif.), providing a platform for mobile and remote use of the device of the present invention, for example, under field conditions and in clinics and hospital wards. Such mobile luminescence detectors can be made, for example, using integrated circuit optical transducers that directly interface with reporter cells, forming “Bioluminescent Bioreporter Integrated Circuits (BBICs) that can be contained within a small (approximate 5 mm²) area and comprise two main components; photodetectors for capturing the bioluminescent reporter cell signals and signal processors for managing and storing information derived from the bioluminescence. If required, remote frequency (RF) transmitters can also be incorporated into the device in general, or into the overall integrated circuit design for wireless data relay. Since all required elements are completely self-contained within the BBIC, operational capabilities are realized by simply exposing the BBIC to the desired test sample. In some embodiments, the system further comprises a display for displaying detected events.

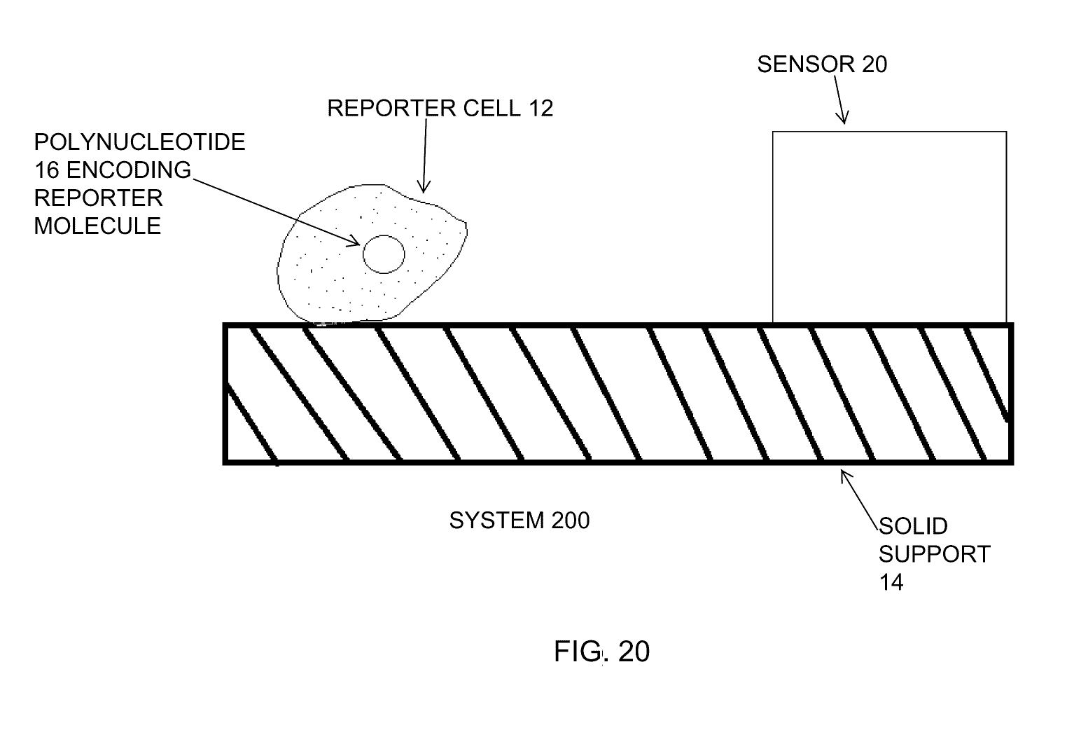

FIG. 20 illustrates an exemplary system 200 of one embodiment of the present invention, having reporter cell 12, which comprises the polynucleotide 16 having the first and second nucleotide sequences as described herein, the reporter cell immobilized on the solid support 14, the device further comprising a sensor 20 for detecting the detectable signal from the reporter molecule. In some embodiments, the sensor 20 is located on, attached to or incorporated into the solid support 14. In other embodiments, the sensor 20 is separate from the solid support—for example, the sensor can be comprised in a docking station into which the solid support is placed, within suitable proximity to the reporter cell, in order to detect the detectable signal.

The system or device can also comprise a sample holder for locating a test sample in sufficient proximity to the reporter cell for an effective amount of the LuxR-like receptor ligand, when present, to contact and bind to the LuxR-like receptor molecule.

The “sample” may be an biological sample either obtained from human, animal or other sources and can be, but is not limited to: respiratory air, sweat, saliva, sputum, blood, plasma, urine, milk (mammary secretion), pleural fluid, cerebrospinal fluid, meningeal fluid, amniotic fluid, lymph, glandular secretions, semen, pus, feces, vomitus, tears, tissue biopsy, cell culture, ambient air, tissue sample obtained from a wound or burn, a swab obtained from the nose, ear and eyes, mouth vagina, wound, burn or any other tissue suspected of having an infection, phlegm and mucus. In order to test infections from lungs such as from cystic fibrosis patients the sample can be mucus but also can be breathe samples exhaled into suitable containers.

The sample may also be non-biological such as medical devices, intubations, catheters etc used in hospital that need to be monitored for P. aeruginosa infections. In some embodiments, the device can be designed to continuously monitor samples of air in the vicinity of the medical device and give a warning once a predefined level of 2AA is reached or exceeded.

The device of the present invention can detect volatile organic molecules that bind to and activate the LuxR-like receptor protein to produce a signal from the reporter cell. Thus, in some embodiments, the sample is not a fluid or solid sample itself but rather either a sample that is gaseous itself (such as breath samples from cystic fibrosis patients) or a gaseous fraction (for example air) that is in direct contact with the sample and to which the volatile organic molecule (such as 2-AA) was released. Thus, in some embodiments, the device is designed so that the reporter cell is positioned to contact a volatile sample, for example, the reporter cell can be in gaseous communication with the test sample. In other embodiments, the reporter cell is in fluid communication with the test sample. As used herein, the phrase “gaseous communication” refers to the placement of the reporter cell so that gas or gases comprising or emanating from the sample can contact the reporter cell. Gaseous communication can include means for preventing dispersion of the gas or gasses of or emitted by the sample, traps for containing and optionally concentrating the gases, heating elements for preventing condensation/phase shift of a gaseous sample, conduits for moving gases from the sample to the reporter cell and the like.

The present inventors have surprisingly uncovered that 2-AA can bind and activate the luxR-like receptor protein, and that 2-AA secreted by P. aeruginosa can be detected by a reporter cell comprising the polynucleotide comprising the first and second nucleic acid sequences of the present invention. Thus, according to some aspects of the invention, there is provided a method of detecting the presence of luxR-like ligands, such as 2-AA in a sample, the method comprising contacting the sample with the device of the invention, wherein detection of a detectable signal above a predetermined level is indicative of the presence of 2-AA (or other luxR-like ligands) in the sample. In some embodiments the luxR-like ligand is 2-AA.

Since 2-AA is secreted by P. aeruginosa, in some embodiments of the invention there is provided a method of detecting a P. aeruginosa infection in a subject, the method comprising contacting a biological sample from the subject with the device of the invention, wherein detection of a detectable signal above a predetermined level is indicative of the presence of a P. aeruginosa infection in the subject.

In some embodiments of the invention there is provided a method of detecting a 2-AA-producing organism in a sample, the method comprising contacting the sample with the device of the invention, wherein detection of a detectable signal above a predetermined level is indicative of the presence of a 2-AA producing organism in the sample.

In some embodiments, the method of detecting luxR-like ligands, such as 2-AA, or detecting a P. aeruginosa infection in a subject, or detecting a 2-AA producing organism in a sample further comprises calibrating the device of the invention with 2-AA standard samples so as to assign amounts or concentrations of 2-AA to values of the detectable signal.

Calibration of the device of the present invention can be performed by contacting the reporter cell(s) of the device with varying concentrations of exogenously added 2-AA and measuring the concentrations and time required for induction of a measurable luminescent response. Standard dilutions of 2-AA are prepared, for example, from 0.001 nM to 100 nM, and spotted on an absorbent substrate, and placed in proximity of the reporter cell in the device, e.g. in a sample holder. Detection of the detectable signal is recorded for example, from time zero and at 30-second to 30-minute intervals, over a predetermined period of time. Data can be plotted as events (e.g. photons) per unit time, over time, and the range of sensitivity of the device for 2-AA can be determined, for example, by selecting a range in which response of the reporter cells to 2-AA is linear with 2-AA concentration. Thus, values of 2-AA concentration, in a predetermined vicinity of the device, can be determined from the record of signal events recorded from the reporter cell. Once the values of the standard are determined, the sample data are analyzed according to the number of detectable events, over time, which occur. The higher the concentration of 2-AA in the sample, or emitted from the sample, the greater the number of events recorded per unit time. Control samples can also be used in calibrating and in actual use of the device of the invention. Positive controls can include, for example, addition of known luxR-like ligands (e.g. 2-AA) to a sample to observe additive signal production and addition of luxR-like receptor or lux pathway antagonists, to verify the specificity of the response. Negative controls can include, but are not limited to introduction of control devices comprising cells deficient in components of the reporter pathway, or comprising different, ligand non-responsive reporter pathways.

In some embodiments, detection of the luxR-1 like ligand (e.g. 2-AA) is performed on a gaseous sample. Gaseous samples can be collected from the vicinity of suspected sources of the luxR-like ligand, for example, in sealed vials, onto absorbent material, such as charcoal, or by concentration and liquefaction. Reconstitution as a gas can be achieved by heating the liquefied sample.

In some embodiments the gaseous sample is collected from the headspace gas of samples. As used herein, the term “headspace gas” refers to any gaseous material above, or surrounding a sample. For solid or liquid samples in closed containers, for example, the headspace gas is that portion of the contents of the container that does not include the solid or liquid sample. Headspace gas of a bacterial culture is the gaseous fraction collecting above the liquid or solid medium in which the bacteria are cultured.

When analyzing a gaseous sample, contact between the sample and the reporter cells should be made under conditions enabling binding of volatile 2-AA to the receptor, activating the LuxR-like receptor, and producing the detectable signal. Prevention of direct contact under such conditions is important for specificity since 2-AA is the only volatile molecule that activates the luxR-like receptor. Non-limiting example of suitable conditions are PBS buffer, approximately pH 8, and exposure from a few minutes to overnight at 37 degrees.

For sampling of wounds, infections or contaminated instruments (e.g. medical devices), soil, waste water and other samples which may need to be sampled in-situ, headspace gas can be sampled by placing the device in maximal proximity (depending on the sensitivity of the reporter cell(s)) to the sample, for example, a few millimeters to a centimeter from a wound or burn suspected for P. aeruginosa infection. In some embodiments, the device of the invention can comprise a means for collecting headspace gas (e.g. suction tube) and thus can sample headspace gas from a remote location in real time. Such a collection method could be particularly suited for monitoring 2-AA and diagnosing P. aeruginosa infection in patients suffering from otitis media, or burn patients, from the exudates, without need for removing a sample. Yet further, the device can comprise a containment for maintaining the gaseous sample in contact with the reporter cells, without loss or dilution of the gasses to the surrounding air.

When testing a gaseous sample, the sample can be the headspace gas from human, animal or other sources and can be, but is not limited to: a gaseous sample such as respiratory air, or the headspace gas from sweat, saliva, sputum, blood, plasma, urine, milk (mammary secretion), pleural fluid, cerebrospinal fluid, meningeal fluid, amniotic fluid, lymph, glandular secretions, semen, pus, feces, vomitus, tears, tissue biopsy, cell culture, ambient air, tissue sample obtained from a wound or burn, a swab obtained from the nose, ear and eyes, mouth vagina, wound, burn or any other tissue suspected of having an infection, phlegm and mucus.

In some embodiments, the device can be used for detection of 2-AA, or P. aeruginosa infection in any chronic inflammation, infection or condition, as well as for detection of contamination of (medical) instruments. 2-AA and P. aeruginosa can be detected by the device in order to detect a disease or pathological condition including, but not limited to chronic otitis media (exudative), supportive acute otitis media, abscess drainage, infected burns, productive cough due to bronchitis, pneumonia, sinusitis, rhino sinusitis, infected catheters including—mechanical ventilation tubes, peripheral and central lines, urinary catheter, infected decubitus ulcers, diabetic ulcers, milk and medical devices. Of particular importance are any wounds, burns, infections or skin lesions in immune compromised individuals.

In a particular embodiment, the subject is suspected suffering from otitis and the biological sample is a sample of otic exudate, headspace gas collected from the otic exudate or headspace gas collected from the affected ear.

The device may also be used for detection of LuxR-like ligand-producing pathogens important for food safety, with appropriate consideration for the nature of the sample under analysis. In general, the reporter cell response may be affected by sample matrix, i.e particulate material generated from sample preparation, in the case of non-gaseous samples. Particulate material may bind reporter cells and cause general quenching of the (light) signal emitted from the reporter cells. Thus, samples may be analyzed to test the effects of sample matrix on the reporter cell assay.

Following detection of 2-AA in a sample with the device of the invention, further confirmation of P. aeroginosa infection or colonization (e.g in a biofilm on medical devices) can be established, for example, by culturing a sample of the suspected material.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

### Growth Conditions

All bacterial strains (Table II) were grown on Luria-Bertani (LB) broth containing 10 g tryptone, 5 g yeast extract and 10 g NaCl in 1 liter of distilled water at 37° C. (except for Agrobacterium tumefaciens A136/pCF218/pMV26, which was grown at 30° C., and for Vibrio fischeri MJ-1, which was grown at 30° C. in LBM (LB+2% NaCl) medium).

TABLE II
Bacterial strains and Plasmids

| | Genotype/relevant characteristics |
|---|---|
| Strain | |
| Vibrio fischeri MJ-1 | Wild type |
| P. aeruginosa PAO1 | Wild type |
| P. aeruginosa PAO1 ΔlasR::Tc^{r} | Mutant deficient in LasR response regulator. |

QS reporter strains

| | |
|---|---|
| Strain/plasmid (antibiotic resistance) | |
| E. coli harboring pSB401(Tc^{r}) | Luminescent strain expressing LuxR of Vibrio |
| | fischeri, activated by C4-HSL, 3-oxo-C6-HSL, C6- |
| | HSL, 3-oxo-C8-HSL and C8-HSL. |
| E. coli JLD271/pAL103 (Tc^{r}) | Luminescent strain expressing LuxR (as pSB401). |
| E. coli JLD271/pAL104 (Tc^{r}) | LuxR-negative strain |
| E. coli pSB536 (Amp^{r}) | Luminescent strain expressing AhyR of Aeromonas |
| | hydrophila, activated by C4-HSL. |
| P. aeruginosa JP2/pKD201(Tmp^{r}) | Luminescent strain expressing native LasR of P. aeruginosa, |
| | activated by 3-oxo-C12-HSL. |
| P. aeruginosa JP2/pKD-rhlA(Tmp^{r}) | Luminescent strain expressing cognate RhlR of P. aeruginosa, |
| | activated by C4-HSL. |
| Salmonella enterica 14028/pBA405E (Tc^{r}) | Luminescent strain expressing cognate SdiA |
| | response regulator, activated by 3-oxo-C6-HSL |
| | and 3-oxo-C8-HSL. |
| Agrobacterium tumefaciens A136/pCF218/pMV26 | Luminescent strain expressing cognate TraR |
| (Tc^{r}) | response regulator, sensitive to C8-HSL, C6-HSL |
| | and 3-oxo-C6-HSL. |
| Burkholderia cenocepacia H111-I/pAS-C8(Gm^{r}) | Fluorescent strain expressing its CepR response |
| | regulator, activated by C8-HSL. |

C4-HSL: N-butanoyl-homoserine lactone;
3-oxo-C6-HSL: N-3-oxo-hexanoyl-homoserine lactone;
C6-HSL: N-hexanoyl-homoserine lactone;
3-oxo-C8-HSL: N-3-oxo-octanoyl-homoserine lactone;
C8-HSL: N-octanoyl-homoserine lactone;
3-oxo-C12-HSL: N-3-oxo-dodecanoyl-homoserine lactone

Effect of Total Volatiles Produced by Pseudomonas aeruginosa on Quorum Sensing Reporter Strains.

For examination of the effect of P. aeruginosa volatiles on various quorum sensing (QS) response regulators, P. aeruginosa PAO1 was inoculated with different QS-reporter strains (Table I) in two separate compartments of bi partite Petri dishes. Such a compartmental inoculation apparatus enabled only the exchange of volatiles between P. aeruginosa culture and the examined reporter strain. For assays evaluating possible antagonism/synergism of P. aeruginosa's volatiles towards QS response regulators, the reporter strains exposed to P. aeruginosa's volatiles were inoculated with their relevant Acyl homoserine lacton (AHL) signaling molecule (Cayman Chemical Company, Ann Arbor, Mich., USA). In these experiments 1 μl of N-3-oxo-dodecanoyl-homoserine lactone (3-oxo-C12-HSL) and N-3-oxo-hexanoyl-homoserine lactone (3-oxo-C6-HSL) was added at a concentration of 1 μM, and 1 μl of N-butanoyl-homoserine lactone (C4-HSL) and N-octanoyl-homoserine lactone (C8-HSL) at a concentration of 100 μM. For assays examining agonistic activity of P. aeruginosa volatiles, both strains were incubated without any addition of exogenous AHL. Following over night incubation, the colonies of the reporter strain were scraped from the agar, re-suspended in phosphate buffer saline (PBS; 0.1 M pH=7.4; 10.9 g 1-1 of Na2HPO4, 3.2 g 1-1 of NaH2PO4 and 9 g 1-1 of NaCl) and measured for luminescence in a 96 well plate using infinite-F200 plate reader (Tecan Trading AG, Switzerland). Relative luminescence was calculated as the luminescence divided by the optical density. Relative green fluorescence produced by B. cenocepacia H111-I/pAS-C8 was measured with an excitation wavelength of 465 nm and an emission wavelength of 535 nm.

Alternatively, P. aeruginosa PA14 was inoculated with several reporter strains in two separate compartments of a bi-partite petri dish. After overnight incubation the culture of the reporter strains was analyzed for luminescence production as described above.

Volatile profiles analysis 500 μl of medium with either PA01 or PA01 ΔlasR in triplicates were diluted with 500 μl of DDW. 5 μl of 1 ppm benzylacetone in MeOH were added as an internal standard, to a final concentration of 0.33 μM or 5 ppb. Stir Bar Sorptive Extraction was carried out using a 1×10 mm PDMS-coated Twister bar (Gerstel GmbH, Mülheim an der Ruhr, Germany), for 8 h. The Twisters were wiped and rinsed with DDW and were subjected to Thermal Desorption coupled to a Programmed-temperature vaporization (PTV) injector (TDU-CIS-4, Gerstel). Desorption was carried out under TDU splitless conditions with 40 ml min−1 He flow, and a temperature gradient of 60° C. min−1 from 20° C. to 170° C. with a 5 mins hold. The PTV inlet was fitted with a quartz wool liner (Gerstel) and kept under −20° C. for the duration of the desorption process, after which a temperature gradient of 12° C. sec−1 ensued, up to 250° C. with a 10 min hold.

A 7890 Gas Chromatograph (GC) coupled to a 5375 Mass spectrometer (MS) (Agilent technologies, Santa Clara, Calif.), fitted with a Rxi-XLB 30×0.25×0.25 Column (Restek, Bellafonte, Pa.) were used to run the analyses. GC oven temperature gradient was 40° C. for 3 minutes then 15° C. min-1 to 280° C. for 5 mins. MS was operated in positive EI scan (40-400 amu) mode, 70 eV energy. Obtained chromatograms were analyzed with Chemstation software (Agilent) and mass spectra were compared to Wiley9/NISTO8 combined mass spectral library (Wiley and Sons, Hoboken, N.J.) and/or NIST11 (NIST, Gaitersburg, Md.). 2-aminoacetophenone (2-AA) and benzylacetone identification was verified with commercials standards (Sigma) for spectra and retention times.

Integration was carried out in Chemstation using chemstation integrator. Areas under the curve (AUCs) were normalized to the AUC of the internal standard.

### Effect of 2-Aminoacetophenone on Specific QS-Reporter Strains

2-AA was applied to various reporter strains in order to evaluate whether it could inhibit or activate different QS response regulators. The reporter strains were grown overnight at 30° C. in LB medium with an appropriate antibiotic and then washed and diluted 1:100 with fresh LB medium, obtaining a concentration of approximately 10⁷ cells ml−1. 100 μl of the cultures were added per well to a 96-wells plate (Corning Inc., NY, USA. Cat. number 356701) in four replicates. Assays for antagonistic/synergistic activity were prepared by the addition of 2-AA together with a specific AHL to the reporter strains cultures. Agonism assay was carried out by the addition of 2-AA to the reporter strain without the addition of any AHL. The negative controls lacked both 2-AA and AHL while the positive controls contained only the appropriate AHL at various concentrations. 2-AA was added for both agonism and antagonism/synergism assays at concentrations of 1, 10, 25, 50, 100 and 500 μM. C4-HSL, C8-HSL and 3-oxo-C12-HSL were added for positive controls at concentrations of 1, 10, 25, 50, 100 and 500 μM, while 3-oxo-C6-HSL was added at 1, 10, 25, 50 and 500 nM. For antagonism/synergism assays C4-HSL, C8-HSL and 3-oxo-C12-HSL were added at concentration of 10 μM, while 3-oxo-C6-HSL was added at 10 nM. 2-AA was added directly to the culture of the reporter strains before dividing it to the wells of the 96-well plate, while one microliter of various AHLs at different concentrations, dissolved in acetonitrile, was placed in the well half an hour prior to the addition of the cultures to allow evaporation of acetonitrile. The bacteria within the plates were then incubated for 24 h at 37° C., except for A. tumefaciens A136/pCF218/pMV26, which was incubated at 30° C. During the incubation, optical density (OD λ=600 nm) and the luminescence or the fluorescence produced by the reporter strains were measured at 30 min intervals using infinite-F200 plate reader (Tecan Trading AG, Switzerland).

The effect of 2-AA in its volatile state was examined as follows: Briefly, 10 nmol of 2-AA and 100 μl of overnight incubated reporter strain were added to 2 opposite sides of bi partite Petri dishes. Alternatively, 1 μg of 2-AA was examined for LuxR activation in a volatile assay. One microliter of 2-AA in a concentration of 1 μg/μl was placed on blank disks (Oxoid, UK) on a cover of a petri dish. Twenty microliters of an overnight grown culture of E. coli/pSB401 were inoculated on the second part of the petri dish. The dishes were sealed and incubated overnight in static conditions at 37° C. Following over night incubation the colonies of the reporter strain was scraped from the agar plate, colonies resuspended, portioned into 96 well plates and relative luminescence was measured as describe above.

Effect of 2-Aminoacetophenone on Vibrio fischeri's LuxR-Regulated Luminescence.

2-Acetoaminophenone was added to V. fischeri in order to verify the activity of 2-AA on QS-regulated traits in a LuxR-harboring wild-type strain. The starters for the experiment were prepared as follow: prior each experiment, a culture from a glycerol stock was inoculated in LBM medium and incubated overnight at 30° C., then diluted 1:1000 and incubated overnight again. The culture was then washed and diluted 1:1000 prior to addition of 25, 50 or 100 μM of 2-AA, or 10 nM of 3-oxo-C6-HSL. Luminescence and absorbance of MJ-1 cultures incubated in 96-well plate was measured as described above. It should be mentioned that in two repeats of the experiment, no luminescence was measured either following the addition of AHL or addition of 2-AA (data not shown).

### Effect of 2-AA Analogs on LuxR

Seven analogs of 2-AA were tested against E. coli/pSB401 and E. coli JLD271/pAL103 in order to evaluate what chemical groups of 2-AA are involved in ligand-receptor interaction. The following compounds were tested: 4-aminoacetophenone, 3-aminoacetophenone, aminoacetophenone, 2-nitroacetophenone, methyl anthranilate, anthranilic acid and 2-aminobenzaldehyde (Sigma, St. Louis, USA). The compounds were applied to the reporter strain in the concentrations of 1-50 μM as describe for 2-AA. Luminescence was measured after 12 h in a plate reader.

### Multiple Sequence Alignment

Multiple sequence analysis (MSA) was done on TraR (PDB code: 1L3L), LasR (PDB code: 2UVO), SdiA (PDB code: 2AVX) and LuxR (Uniprot entry: P12746), using T-Coffee (see www dot tcoffee dot vital-it dot ch apps tcoffee index). In addition, portions of the LuxR response regulators of the following species were aligned with LuxR of Vibrio fischeri (accession number CAA68561.1)(SEQ ID NO: 1): Aliivibrio logei (AAQ90213.1) (SEQ ID NO: 2), Vibrio mimicus (AAQ90214.1) (SEQ ID NO: 3), Photobacterium leiognathi (AAQ90227.1) (SEQ ID NO: 4) and Vibrio parahaemolyticus (AAQ90194.1) (SEQ ID NO: 5).

### Homology Model Construction

LuxR (SEQ ID NO: 6) (Uniprot entry: P12746) was aligned with TraR (PDB code: 1L3L) using the T-Coffee algorithm. A model of LuxR was created using the Modeller protocol (1) as implemented in Discovery Studio 4.0 (DS 4.0, Accelrys). Twenty models were generated and model quality was assessed using the protein report tool (DS 4.0) and the model with the best score was chosen for further refinement, which included minimization. Default protocol settings were used.

### Identification of a Binding Site in the Model

Binding site was defined using ‘define binding site’ protocol in DS 4.0. This protocol is based on an ‘eraser and flood-filling grid algorithm’, where binding sites are identified based on the shape of the receptor. The best scored site was determined as the binding site for the generated model. Default algorithm settings were used.

### Ligand Docking

Ligands were prepared using ‘prepare ligands’ protocol and conformations were generated using ‘generate conformations’ protocol, both as implemented in DS 4.0. Docking of the ligands was performed using CDocker protocol (DS 4.0). Default protocols settings were used.

Analysis of Pus Samples from Patents Exhibiting Symptoms of Otitis Externa

Pus samples obtained from 7 patients (2-80 years old) suffering from otitis externa were analyzed. In each sample the presence of P. aeruginosa was examined by three separate methods:

(i) one portion of the sample was used for routine culturing swab assays;

(ii) a second portion of the sample was subjected to the dynamic head space analyses using GC for 2-AA detection. For dynamic headspace analysis the pus was suspended in standard saline solution (0.9% NaCl) and put in 20 mm headspace vials. The headspace vial was agitated and incubated in 60° C. for 10 minutes. The headspace was continuously collected for 1 hr using 500 ml of Helium at 20 ml/min, unto a Tenax TA tube. The Tenax tube was desorbed in a Thermal desorption unit (Gerstel TDU) for 4 minutes at 210 C in splitless mode and vapors subsequently trapped in a PTV injector (Gerstel CIS4) that was kept in −70 C. After desorption was complete, the PTV was heated at 12 C/sec to 300 C and was held there for 4 minutes. The GC Column was Restek Rxi-XLB medium polarity column, 30×0.250×25. He flow was 1.1 ml/min, Oven program was 40° C. for 3 min then 12.5° C./min to 300° C. for 3 min. Mass Spectra acquisition was done in SIM mode (Selective Ion Monitoring), for masses 92.0; 120.0; 135.0;

(iii) The third portion of the sample was examined with the E. coli/pSB401 reporter strain for 2-AA activity against LuxR response regulator. For the LuxR activation assay, the pus sample was transferred to sterile 1.5 ml centrifuge tube. Twenty microliters of overnight grown culture of E. coli/pSB401 reporter strain were placed on 250 μl of agar that was solidified on the inner part of the centrifuge tube cap. The tube was then closed and incubated overnight in static conditions at 37° C. There was no contact between the pus sample and the reporter strain and activation occurred only through volatile emission from the pus. Following incubation, the colony of the reporter strain was transferred into 100 μl of PBS in 96-well plate. Luminescence and the absorbance of the suspended colonies were measured in a plate reader.

### RESULTS

### Effect of P. aeruginosa's Volatiles on Luminescence-Based QS Reporter Strains

P. aeruginosa PA14 was inoculated with several reporter strains in two separate compartments of a bi-partite Petri dish. Such a compartmental inoculation of the tested and reporter strains enables only exchange of volatiles between them. A significant induction of luminescence was detected in E. coli/pSB401 (reporter strain (FIG. 1), indicating that volatile substances of P. aeruginosa could induce quorum sensing (QS) cell-to-cell communication regulatory pathways, presumably those mediated by of C6- and C8-Homoserine lactones (HSL) signal molecules.

In another series of experiments, in order to identify potential volatile substances that can act as either QS agonists or antagonists, the effect of total volatiles produced by P. aeruginosa on several QS bioreporters was observed, using bi-partite Petri dishes that have separate compartments but a joint headspace allowing only the exchange of volatile substances. The bioreporters used in this study respond to various Acyl homoserine lactone (“AHL” or “HSL”) molecules ranging in carbon chain length from four to 12 carbons. These molecules are the most common QS signaling molecules used by Gram negative bacteria for communication. The reporter strains used in this study with their designated response regulators are summarized in Table I.

Volatiles of P. aeruginosa PAO1 strain significantly induced a positive luminescence response in the Escherichia coli/pSB401 reporter strain, regulated by Vibrio fischeri LuxR response regulator (P<0.05; ANOVA on Ranks and Student-Newman-Keuls post hoc test) (FIG. 2). Moreover, the effect of P. aeruginosa's volatiles was synergistic to the induction obtained by 1 pmol of N-3-oxo-hexanoyl-homoserine lactone (3-oxo-C6-HSL) (FIG. 2). Notably, P. aeruginosa's volatiles did not affect any of the other examined response regulators (data not shown), nor did they act as antagonists to LuxR (FIG. 2), indicating that certain compound/s from P. aeruginosa's total volatiles can specifically activate the LuxR response regulator.

P. aeruginosa possesses three different QS systems that are crucial for its full virulence and persistence within the host. Two systems, las and rhl, are activated by N-3-oxo-dodecanoyl-homoserine lactone and N-butanoyl-homoserine lactone, respectively. The third system, mvfR, is activated by the quinolones signals 4-hydroxy-2-heptylequinolone and Pseudomonas quinolone signal (PQS).

Overall, more than 10% of P. aeruginosa's genome is under the regulation of QS. In order to determine whether a QS mutant could maintain its ability to activate the LuxR reporter strain, response of a ΔlasR mutant, deficient in the production of the LasR response regulator was tested. This mutant was chosen since the three QS systems of P. aeruginosa are hierarchically organized such that the las QS system is dominant over the rhl and PQS.

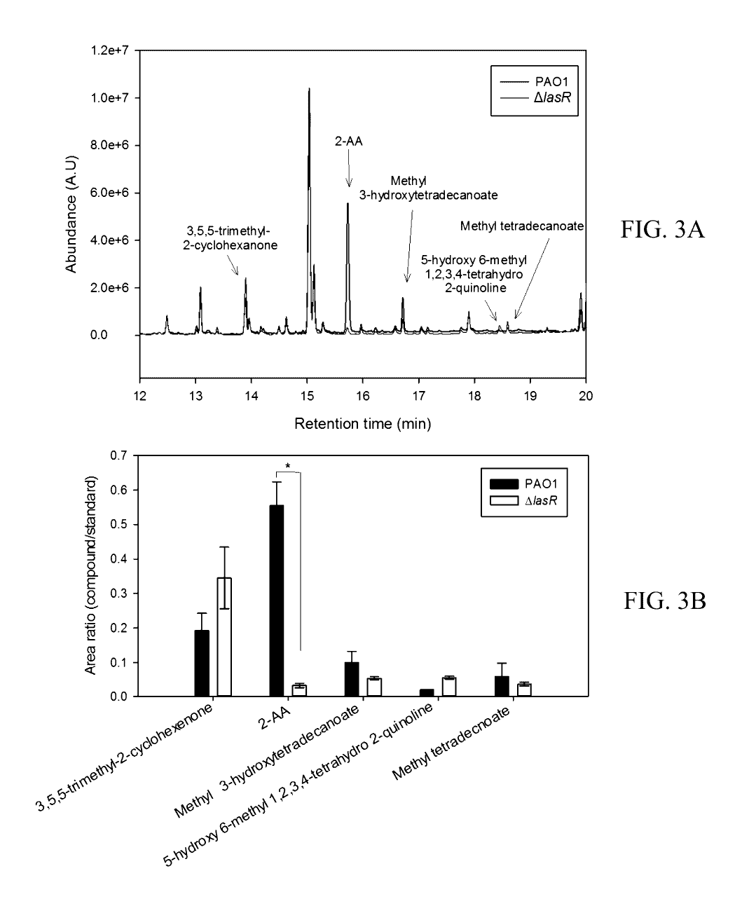

In contrast to the WT strain, total volatiles of the ΔlasR mutant did not activate the LuxR response regulator (data not shown). In order to identify the volatile substance/s responsible for the activation of the LuxR response regulator by P. aeruginosa, a comparative gas-chromatograph mass-spectrometer (GC/MS) analysis of the ΔlasR and WT strains was performed. As seen in FIGS. 3A and 3B, the main volatile missing from the volatiles profile of mutations in lasR was 2-aminoacetophenone (2-AA).

Effect of 2-AA on Luminescence-Based QS Reporter Strains

2-AA is predominant among the volatile profile of P. aeruginosa. In order to determine whether the induction of QS-regulated luminescence was caused specifically by 2-AA, luminescence of various QS-reporter strains was measured upon addition of 2-AA (50 μM). Results show that the E. coli/pSB401 reporter, which is sensitive mainly to C6-HSL exhibited a significant two-order-of-magnitude induction of luminescence upon exposure to 2-AA (FIG. 4).

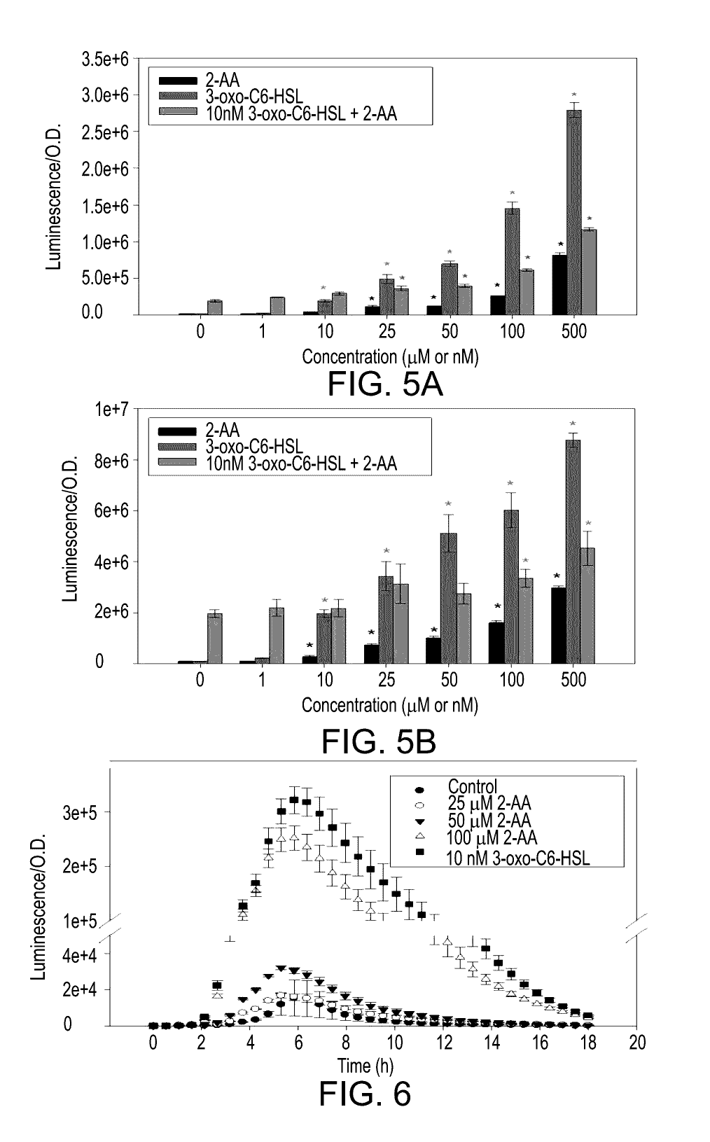

In another series of experiments, different concentrations of synthetic 2-AA, either as volatiles or in a dissolved state, were added to cultures of E. coli/pSB401 and E. coli JLD271/pAL103 reporter strains. 2-AA was able to significantly induce the LuxR-regulated luminescence of the reporter strains when applied both as a liquid (FIGS. 5A and 5B) and as a volatile (data not shown) (p<0.05; ANOVA and Dennett post hoc test, and student's t-test, respectively).

Similarly to the effect of P. aeruginosa's total volatiles, addition of 2-AA to the biosensors inoculated with a fixed concentration of AHL (10 nM) further induced the LuxR-regulated luminescence as compared to the value measured with 10 nM of AHL without 2-AA (FIGS. 5A and 5B, green bars).

However, there was a difference in the range of concentrations having a synergistic effect on E. coli/pSB401 biosensor (25-500 μm) compared to E. coli JLD271/pAL103 biosensors (100 and 500 μm). In order to verify that the observed induction of luminescence by 2-AA occurred via LuxR activation, 2-AA was applied to an E. coli JLD271/pAL104 reporter strain, which harbours the same plasmid as pAL103 but lacks the gene encoding LuxR.

No effect of 2-AA on the LuxR-negative reporter was observed, suggesting that indeed 2-AA interacts directly with the LuxR receptor (data not shown). Although volatiles of P. aeruginosa activated only the LuxR response regulator, possible cross reaction of the synthetic 2-AA compound with the additional bioreporter strains described above was investigated. Similar to the results obtained with total volatiles of P. aeruginosa, 2-AA did not induce the activity of P. aeruginosa cognate QS receptors, RhlR and LasR (data not shown). 50 and 100 μM of 2-AA slightly inhibited (20 and 16%, respectively) RhlR-regulated luminescence in the presence of AHL, however, 2-AA also slightly inhibited luminescence in absence of AHL to the same level (20%), implying that this inhibition is not via ligand-response regulator interaction. 500 μM of 2-AA exhibited more significant inhibition (decrease of 40%) towards both LasR- and RhlR-regulated luminescence. According to the O.D. measurements, the observed decrease in luminescence was not due to growth inhibition. Thus, while it is feasible that extremely high concentrations (500 μM) of 2-AA directly inhibit LasR and RhlR, such concentrations are unphysiological high and are likely biologically irrelevant. Notably, 2-AA did not exhibit any significant activation of any of the other response regulators examined in this study (i.e. TraR, SdiA, CepR, AhyR and AhlR) (data not shown).

The above results show that 2-AA can activate the luxR response regulator in E. coli based biosensor strains. To fully evaluate the biological significance of this finding the activity of 2AA on the LuxR-regulated natural luminescence of wild type V. fischeri MJ-1 was examined. Without addition of exogenous HSL, wild type V. fischeri MJ-1 exhibited relatively low levels of luminescence. Nevertheless, addition of 10 nM of 3-oxo-C6-HSL resulted in a significant increase in the luminescence (FIG. 6). Significantly, addition of 2-AA to the wild-type V. fischeri MJ-1 also significantly increased luminescence, in a dose dependant manner, similarly to the activation of the LuxR-harboring reporter strains.

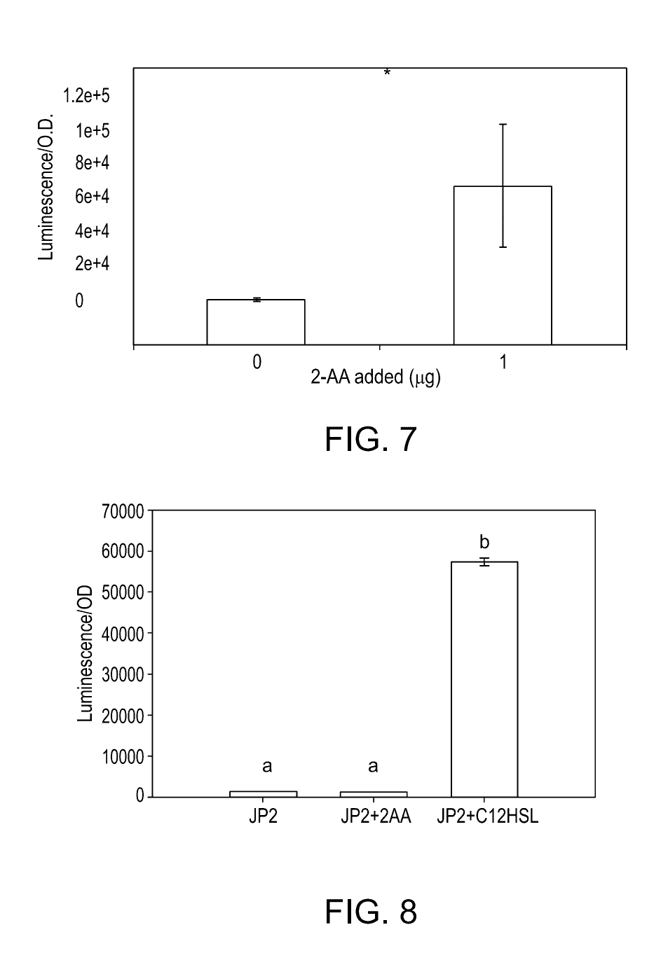

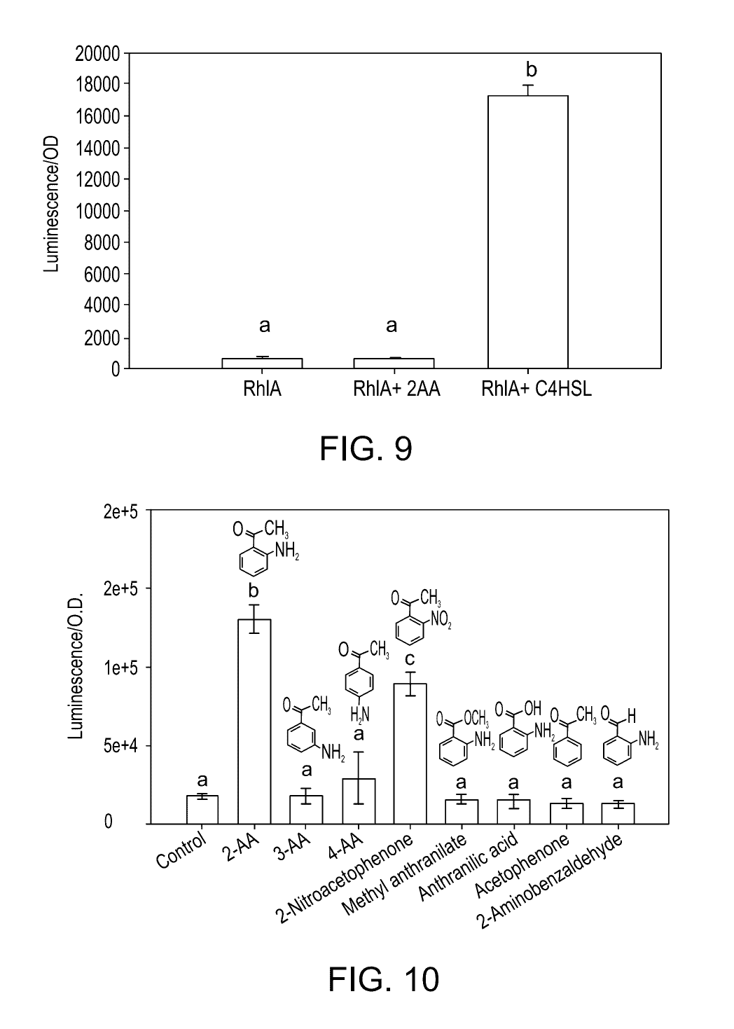

Induction of luminescence was also obtained when 2-AA was applied as a volatile, at 1 μg, to a bipartite Petri dish, opposite inoculated E. coli/pSB401 (FIG. 7). Concentration of 2-AA used in these experiments are consistent with concentration of 2-AA in cultures of P. aeruginosa (5 μg/ml; 37 μM). Notably, the addition of 2-AA had no effect on luminescence of the PAO-JP2/pKD201 strain, which is sensitive to C12-HSL (FIG. 8), and on the P. aeruginosa/RhlA receptor, which is sensitive to C4-HSL (FIG. 9). These results indicate that 2-AA could specifically mimic C6 and C8 chains of HSLs.

Structural Analysis of 2-AA and in-Silico Docking

AHLs (also known as HSLs) and 2-AA are quite different in structure, thus the nature of the interaction between AHL-binding LuxR and 2-AA was not clear. In order to better understand the apparent specificity and interaction of 2-AA with the AHL-binding receptor, the effect of several 2-AA analogues (4-aminoacetophenone, 3-aminoacetophenone, acetophenone, 2-nitroacetophenone, methyl anthranilate, anthranilic acid and 2-aminobenzaldehyde) on luminescence of the E. coli/pSB401 reporter strain was examined (FIG. 10). These analogues either have their amine-group in alternate positions, or a substitution in the ketone group. Deletion or translocation of the amine group to third and fourth positions, as well as substitution of the ketone group with a carboxylic acid, an ester or an aldehyde completely abolished the induction of luminescence exhibited by the reporter strain (P<0.05; ANOVA and Student-Newman-Keuls post hoc test) (FIG. 10). Substitution of the amine group with a nitro group partially reduced LuxR activation compared to 2-AA, indicating that 2-nitroacetophenone could also activate LuxR, but to a lesser extent than 2-AA. Significantly, no dose dependency or synergistic effects in the presence of AHL were observed with 2-nitroacetophenone. Further, none of the analogues, except for 2-nitroacetophenone, exhibited either significant inducing activity in absence of exogenous AHL, or synergistic/antagonistic activities in presence of AHL (HSL) (data not shown). Without wishing to be limited to a single hypothesis, these results suggest that the presence of the ketone group and the position of the amine group are crucial factors in LuxR-2-AA interactions and activation.

To further investigate the interaction of LuxR with 2-AA, an in silico docking analyses of 2-AA and LuxR's cognate ligand, 3-oxo-C6-HSL, into a LuxR model was undertaken. Overlap of the docked 2-AA and 3-oxo-C6-HSL revealed a similar position of the 2-AA ring and the ring of 3-oxo-C6-HSL within the binding pocket of LuxR (data not shown). While not wishing to be limited to a single hypothesis, the results of the AHL docking suggest that Trp66, Asp79 and Tyr70 are the crucial residues in AHL-LuxR interactions by interacting with AHL via hydrogen bonds (FIG. 11A). In addition to these interactions, hydrophobic interactions with Pro48, Met51, Ile56, Ile76, and Val82 may act to stabilize the carbon chain. Docking of 2-AA into LuxR model indicated that some of the LuxR conserved residues that participate in 3-oxo-C6-HSL interactions (Trp66, Tyr70 and Asp 79) also play a role in the interactions between 2-AA (blue) and the receptor (FIG. 11B). One possibility is that Tyr70 and Asp79 could form hydrogen bonds with the amine group, Trp66 with the carbonyl group. Additionally, Tyr62, Leu118, Ala139, Ile46, Ile81 were suggested to be involved in hydrophobic interaction. The combination of all five residues forming the hydrophobic interactions is not conserved among different QS receptor binding sites and is unique to LuxR compared to SdiA, LasR and TraR (data not shown), consistent with the specificity of 2-AA towards LuxR.

Taken together, the results provided herein indicate that 2-AA, a low molecular weight volatile compound produced by P. aeruginosa in relatively high amounts (up to 80 μM) and suspected important in the persistence of the pathogen and its interaction with the host is a specific activator of the LuxR response regulator. As apparent from FIGS. 4 through 9, the affinity of 3-oxo-C6-HSL towards LuxR is approximately three orders of magnitude higher than that of 2-AA. The physiological relevance of the different affinities may, nevertheless, lie in the differences in physiological concentrations of the two volatiles: the concentrations of 3-oxo-C6-HSL measured in bacterial cultures of Vibrio spp. varies between 1 to 10 nM, whereas those of 2-AA in P. aeruginosa cultures reached 80 μM.

2-AA did not activate the QS receptors of P. aeruginosa. While not wishing to be limited to a single hypothesis, it is conceivable that 2-AA might serve as an inter-species signal, activating QS systems in other bacteria. 2-AA has been detected in total volatiles of several bacterial species inhabiting various environments ranging from the human body to marine sediments. BLAST analysis of LuxR homologs from other bacterial species revealed that several bacterial species other than V. fischeri, such as Aliivibrio logei, Vibrio mimicus, Photobacterium leiognathi and Vibrio parahaemolyticus, possess highly similar LuxR homologs that include all the residues that were found to interact with 2-AA but are lacking in the non-reactive SdiA, TraR and LasR receptors (FIG. 12).

Mass Spectrometry Analysis of 2-AA:

Mass spectrometry (MS) analysis of 2-AA standard was performed for calibration purposes. TIC—Total Ion Chromatogram measurements (FIG. 13A) showed a peak in retention time of 17.567, and the Selected Ion Monitoring spectrum (SIM) (FIG. 13B) showed the three ions in the correct ratio −70:100:50 for 135, 120 and 92 respectively.

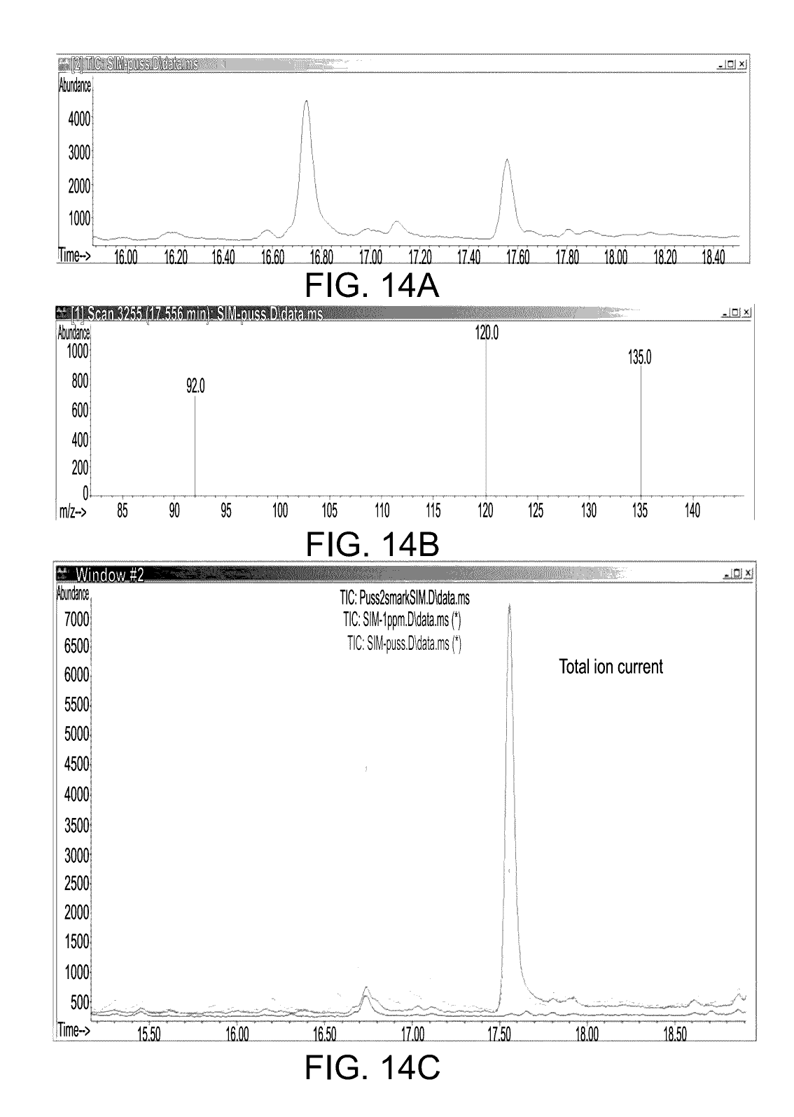

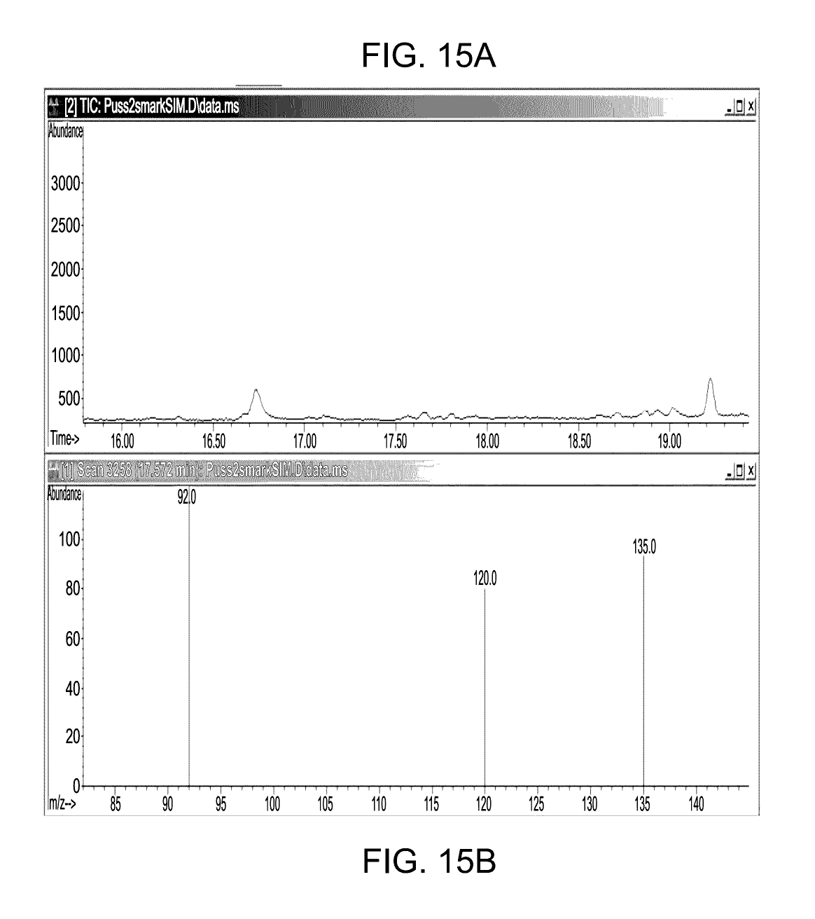

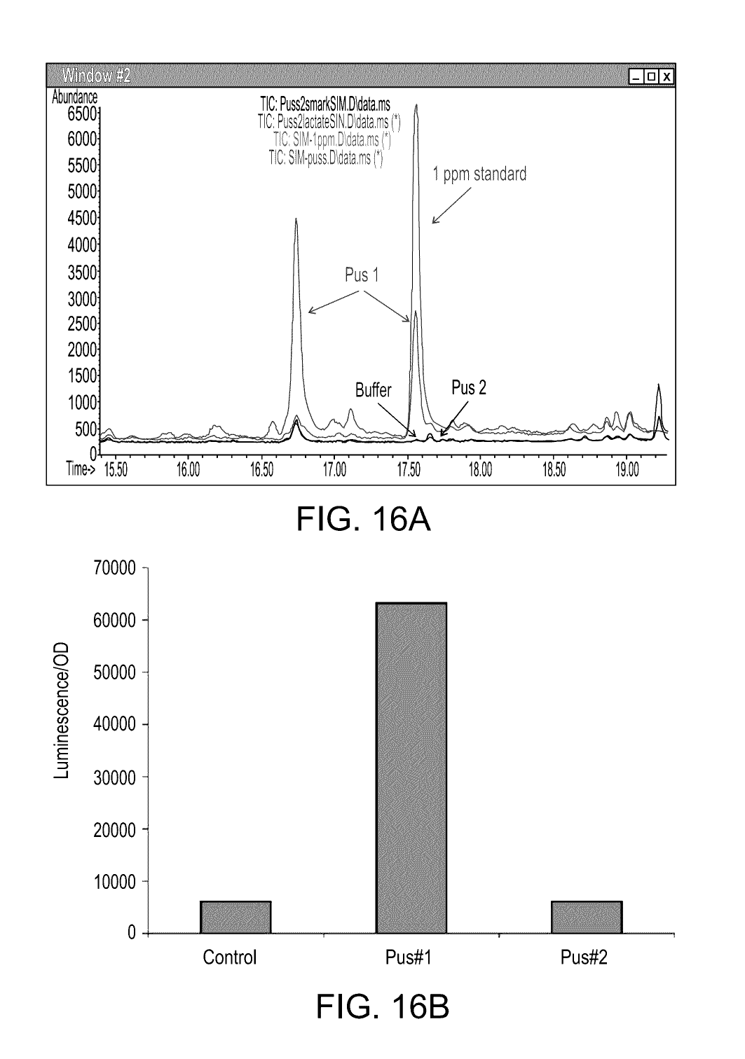

After obtaining an accurate 2-AA MS signature, TIC and SIM analysis were performed on pus obtained from patients exhibiting sever outer ear infections. Pus samples were concomitantly analyzed for their ability to activate luminescence in the reporter strain and were sampled for culture tests indicative of the presence of P. aeruginosa. Two samples were used: pus sample number 1, which was positive for P. aeruginosa in culture test, and pus sample number 2, which was negative for P. aeruginosa in culture test (data not shown). TIC and SIM analysis of pus sample number 1 identified 2-AA in this sample (FIG. 14C) and the reporter strain also produced a luminescence signal upon exposure to the volatile emitted from this pus sample (FIG. 16B). Conversely, TIC and SIM analysis could not detect 2-AA in pus sample number 2 (FIGS. 15A and 15B, and 16A). The reporter strain did not produce a luminescence signal upon exposure to the volatile emitted from pus sample number 2 as well (FIG. 16B).

In addition to the samples analyzed in FIGS. 16A and 16B, five more samples of pus from patients of ages ranging from 2-80 years were analyzed. All samples gave negative results in culture, TIC and biosensor analyses, indicating that these patients were not infected by P. aeruginosa.

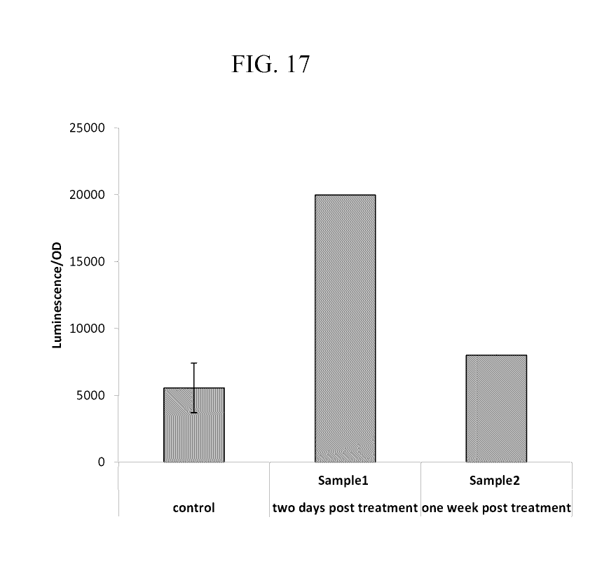

Analysis was also carried out on external wounds treated in horses. Two tissue samples were collected from the digital flexor tendon sheath. Samples from two wounds were examined. Samples from two days (wound number 1, sample number 1) and one week after they started treatment (wound number 2, sample number 2) (no samples were provided at time zero before treatment commenced) were analyzed. Before treatment commenced both wounds were positive for P. aeruginosa bacteria in a culture assay. Samples taken from wounds at the end of the treatment (10 days) were negative for P. aeruginosa bacteria in culture assays. FIG. 17 shows that sample one, which corresponds to 2 days of antibiotic treatment, was positive for 2-AA, whereas sample number two was negative for 2-AA, corresponding to the completion of the antibiotic treatment and anti-bacterial effect.

Taken together, these results indicate that 2-AA can serve as an accurate biomarker for P. aeruginosa infections, for example, of the outer ear (otitis externa), and that a bacterial reporter strain expressing a luxR receptor fused to a reporter gene, can be useful as a simple, accurate and sensitive diagnostic tool for detection of P. aeruginosa infections.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A device, comprising a reporter cell comprising a polynucleotide comprising a first nucleic acid sequence encoding a reporter molecule capable of producing a detectable signal and a second nucleic acid sequence comprising a luxR-like receptor binding element for regulating transcription of said reporter molecule, wherein said reporter cell is attached to a solid support or encapsulated within an encapsulation matrix attached to a solid support.

2. The device of claim 1, wherein said cell further comprises a third nucleic acid sequence encoding a luxR-like receptor capable of binding 2AA.

3. The device of claim 2, wherein said polynucleotide comprises said first nucleic acid sequence, said second nucleic acid sequence and said third nucleic acid sequence.

4. The device of claim 2, wherein said third nucleic acid sequence is comprised on a polynucleotide distinct of said polynucleotide.

5. The device of claim 2, wherein said luxR-like receptor is foreign to the cell.

6. (canceled)

7. The device of claim 2, wherein said cell is devoid of endogenous luxR-like expression.

8. The device of claim 2, wherein said cell is devoid of endogenous luxR-like agonists or antagonists or both.

9. The device of claim 8, wherein said cell is devoid of luxI expression.

10-11. (canceled)

12. The device of claim 1, wherein said reporter molecule is a reporter polypeptide.

13. The device of claim 12, wherein said reporter polypeptide is luciferase.

14. The device of claim 13, wherein said first nucleic acid sequence comprises a luxCDABE gene cluster.

15. The device of claim 1, wherein said reporter cell is selected from the group consisting of a bacterial cell, a fungal cell, a plant cell, an algal cell and an animal cell.

16-21. (canceled)

22. A method of detecting 2AA or derivatives thereof in a biological sample, comprising contacting said sample with the device of claim 2, wherein presence of said detectable signal is indicative of 2-AA in said biological sample.

23. A method of diagnosing a Pseudomonas aeruginosa infection in a subject, comprising contacting a biological sample of the subject with the device of claim 2, wherein detection of said signal above a predetermined level is indicative of the presence of a Psuedomonas aeruginosa infection in said sample.

24. A method of detecting the presence of a 2AA-producing organism in a test sample, comprising contacting said sample comprising said organism with the device of claim 2, wherein the presence of said detectable signal is indicative of the presence of a 2AA-producing organism in said test sample.

25. The method of claim 22, further comprising calibrating the device with 2 AA standard samples so as to assign amounts or concentrations of 2AA to values of the detectable signal.

26-28. (canceled)

29. The method of claim 22, wherein said biological sample is a gaseous sample.

30. (canceled)

31. The method of claim 29, wherein said gaseous sample comprises headspace gas of a suspected bacterial population.

32. (canceled)

33. The method of claim 24, wherein said 2AA-producing organism is Pseudomonas aeruginosa and wherein said amount or pattern of said 2AA detected is indicative of a Pseudomonas aeruginosa infection.

34-35. (canceled)

36. A system for detecting a Pseudomonas aeruginosa infection in a subject, the system comprising the device of claim 2, and a sensor for detecting said detectable signal, optionally a display for displaying an event of detection of said detectable signal and optionally a sample holder in fluid or gaseous communication with the reporter cell, for holding a biological sample from said subject for detection.

37-38. (canceled)


SEQUENCE LISTING (46 sequences)

SEQ ID NO: 1 - protein, 158 aa, Vibrio fischeri
MKNINADDTYRIINKIKACRAYDINQCLSDMTKMVHCEYYLTLAIIYPHSMVKSDISILDNYPKKWRQYYDDANLIKYDPIVDYSNSNHSPINWNIFENNAVNKKSPNVIKEAKTSGLITGFSFPIHTANNGFGMLSFAHSEKDNYIDSLFLHACMNI

SEQ ID NO: 2 - protein, 158 aa, Aliivibrio logei
MKDINADDTYRIINKIKACRSNNDINQCLSDMTKMVHCEYYLLAIIYPHSMVKSDISILDNYPKKWRQYYDDANLIKYDPIVDYSNSNHSPINWNIFENNAVNKKSPNVIKEAKTSGLITGFSFPIHTANNGFGMLSFAHSEKDNYIDSLFLHACMNI

SEQ ID NO: 3 - protein, 158 aa, Vibrio mimicus; identical to SEQ ID NO: 2

SEQ ID NO: 4 - protein, 158 aa, Photobacterium leiognathi; identical to SEQ ID NO: 2

SEQ ID NO: 5 - protein, 158 aa, Vibrio parahaemolyticus
MKDINADDTYRIINKIKRCRSNKDINQCLSDMTKMVHCEYYLLAIIYPHCMVKSDISIVDNYPKKWRQYYDDANLIKYDPIVDYSNSNHSPINWNIFENNAVNKKSPNVIKEAKSSGLITGFSFPIHTANNGFGMLSFAHSEKDNYIDSLFLQACMNI

SEQ ID NO: 6 - protein, 250 aa, Vibrio fischeri
MKNINADDTYRIINKIKACRAYDINQCLSDMTKMVHCEYYLTLAIIYPHSMVKSDISILDNYPKKWRQYYDDANLIKYDPIVDYSNSNHSPINWNIFENNAVNKKSPNVIKEAKTSGLITGFSFPIHTANNGFGMLSFAHSEKDNYIDSLFLHACMNIPLIVPSLVDNYRKINIANNKSNNDLTKREKECLAWACEGKSSWDISKILGCSERTVTFHLTNAQMKLNTTNRCQSISKAILTGAIDCPYFKN

SEQ ID NO: 7 - protein, 232 aa, Aliivibrio logei
MGMKDINADDTYRIINKIKACRSNNDINQCLSDMTKMVHCEYYLLAIIYPHSMVKSDISILDNYPKKWRQYYDDANLIKYDPIVDYSNSNHSPINWNIFENNAVNKKSPNVIKEAKTSGLITGFSFPIHTANNGFGMLSFAHSEKDNYIDSLFLHACMNIPLIVPSLVDNYRKINIANNKSNNDLTKREKECLAWACEGKSSWDISKILGCSVRTVTFHLTNAQMKLNTTNR

SEQ ID NO: 8 - protein, 224 aa, Vibrio mimicus
MGMKDINADDTYRIINKIKACRSNNDINQCLSDMTKMVHCEYYLLAIIYPHSMVKSDISILDNYPKKWRQYYDDANLIKYDPIVDYSNSNHSPINWNIFENNAVNKKSPNVIKEAKTSGLITGFSFPIHTANNGFGMLSFAHSEKDNYIDSLFLHACMNIPLIVPSLVDNYRKINIANNKSNNDLTKREKECLAWACEGKSSWDISKILGCSERTVTFHLTNAQ

SEQ ID NO: 9 - protein, 216 aa, Photobacterium leiognathi
  misc_feature (172)..(172): Xaa can be any naturally occurring amino acid
MGMKDINADDTYRIINKIKACRSNNDINQCLSDMTKMVHCEYYLLAIIYPHSMVKSDISILDNYPKKWRQYYDDANLIKYDPIVDYSNSNHSPINWNIFENNAVNKKSPNVIKEAKTSGLITGFSFPIHTANNGFGMLSFAHSEKDNYIDSLFLHACMNIPLIVPSLVDNYXKINIANNKSNNDLTKREKECLAWACEGKSSWDSSKILGCSERGG

SEQ ID NO: 10 - protein, 208 aa, Vibrio parahaemolyticus
  misc_feature (183)..(183): Xaa can be any naturally occurring amino acid
MGMKDINADDTYRIINKIKRCRSNKDINQCLSDMTKMVHCEYYLLAIIYPHCMVKSDISIVDNYPKKWRQYYDDANLIKYDPIVDYSNSNHSPINWNIFENNAVNKKSPNVIKEAKSSGLITGFSFPIHTANNGFGMLSFAHSEKDNYIDSLFLQACMNIPLIVPSLVDNYRKINIANNKSNXDLTKREKECLAWACEGKSSWDISKI

SEQ ID NO: 11 - DNA, 1866 bp, Vibrio fischeri
  misc_feature (1203)..(1203): n is a, c, g, or t
aagctttacttacgtacttaatttttaaagtatgggcaatcaattgctcctgttaaaattgctttagaaatactttggcagcggtttgttgtattgagtttcatttgcgcattggttaaatggaaagtgacagtacgctcactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtgttaaataatattcacaatgtaccattttagtcatatcagataagcattgattaatatcatatgctctacaagctttaattttattaattattctgtatgtgtcgtcggcatttatgtttttcatacccatctctttatccttacctattgtttgtcgcaagttttgcgtgttatatatcattaaaacggtaatggattgacatttgattctaataaattggatttttgtcacactattgtatcgctgggaatacaattacttaacataagcacctgtaggatcgtacaggtttagcgaagaaaatggtttgttatagtcgaataaacgcaagggaggttggtatgactataatgataaaaaaatcggattttttggcaattccatcggaggagtataaaggtattctaagtcttcgttatcaagtgtttaagcaaagacttgagtgggacttagttgtagaaaataaccttgaatcagatgagtatgataactcaaatgcagaatatatttatgcttgtgatgatactgaaaatgtaagtggatgctggcgtttatnacctacaacaggtgattatatgctgaaaagtgtttttcctgaattgcttggtcaacagagtgctcccaaagatcctaatatagtcgaattaagtcgttttgctgtaggtaaaaatagctcaaagataaataactctgctagtgaaattacaatgaaactatttgaagctatatataaacacgctgttagtcaaggtattacagaatatgtaacagtaacatcaacagcaatagagcgatttttaaagcgtattaaagttccttgtcatcgtattggagacaaagaaattcatgtattaggtgatactaaatcggttgtattgtctatgcctattaatgaacagtttaaaaaagcagtcttaaattaatgttgttaaatcattaatttattttaaatactaagtatattatagggaaaataatgaataaatgtattccaatgataattaatggaatgattcaagattttgataattatgcatataaagaagttaaactaaataatgataatagagtaaaattatctgtcattactgaaagttcagtttcaaaaacattaaatatcaaagatagaattaatctaaatttaaatcagattgtgaattttttatataccgttggtcaacgatggaaaagtgaagaatataatcggcgacgaacctat

SEQ ID NO: 12 - DNA, 1228 bp, Aliivibrio logei
cagcggttagttgtattgagtttcatttgcgcattggttaaatggaaagtgacagtacgcacactgcagcctaatatttttgaaatatcccaagagctttttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaagatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattattctgtaagtgtcgtcggcatttatgtctttcatacccatctctttatccttacctattgtttgtcgcaagttttgcgtgttatatatcattaaaacggtaatggattgacatttgattctaataaattggatttttgtcacactattgtatcgctgggaatacaattacttaacataagtacctgtaggatcgtacaggtttacgcaagaaaatggtttgttatagtcgactaaacgcaagggaggttggtatgactataatgataaaaaaatcggattttttggcaattccatcggaggagtataaaggtattctaagtcttcgttatcaagtgtttaagcaaagacttgagtgggacttagttgtagaaaataaccttgaatcagatgagtatgataactcaaatgcagaatatatttatgcttgtgatgatactgaaaatgtaagtggatgctggcgtttattacctacaacagggtgattatatgctgaaaatgtttttcctgaattgcttggtcaacagagtgctcccaaagatcctaatatagtcgaattaagccgttttgct

SEQ ID NO: 13 - DNA, 1144 bp, Vibrio mimicus
tttgcgcattggttaaatggaaagtgacagtacgctcactgcagcctaatatttttgaaatatcccaagagctctttccttcgcatgcccacgctaaacattctttttctcttttggttaaatcgttgtttgatttattatttgctatatttatttttcgataattatcaactagagaaggaacaattaatggtatgttcatacacgcatgtaaaaataaactatctatatagttgtctttctctgaatgtgcaaaactaagcattccgaagccattattagccgtatgaatagggaaactaaacccagtgataagacctgatgttttcgcttctttaattacatttggagattttttatttacagcattgttttcaaatatattccaattaattggtgaatgattggagttagaataatctactataggatcatattttattaaattagcgtcatcataatattgcctccattttttagggtaattatctagaattgaaatatcagatttcaccatggaatgaggataaatgatcgcgagtaaataatattcacaatgtaccattttagtcatatcagataagcattgattaatatcattattgcttctacaagctttaattttattaattattctgtaagtgtcgtcggcatttatgtctttcatacccatctctttatccttacctattgtttgtcgcaagttttgcgtgttatatatcattaaaacagtaatagattgacatttgattctaataaattggatttttgtcacactattgtatcgcttgaaatacaattgtttaacatgagtgcctgtaggatcgtacaggtttacgtaagaaaatggtttgttatagtcgattaaacgcaagggaggttggtatgactataatgataaaaaaatcggattttttggcaattccatcggaggagtataaaggtattctaagtcttcgttatcaagtatttaagcaaagacttgagtgggacttagttgtagaaaataatcttgaatcagatgagtatgataactcaaatgcagaatatatttatgcttgtgatgatactgaaaatgtaagtggatgctggcgtttattacctacaacaggtgatattatgctgaaaagtgtttttcctgaat

SEQ ID NO: 14 - DNA, 1030 bp, Photobacterium leiognathi
  misc_feature (137)..(137): n is a, c, g, or t
agtgacccccacgctcactgcagcctaatatttttgaactatcccaagagctttttccttcgcatgcccacgctaaacattctttttctcttttggttaaatcgttgtttgatttattatttgctatatttattttncnataattatcaactagagaaggaacaattaatggtatgttcatacacgcatgtaaaaataaactatctatatagttgtctttttctgaatgtgcaaaactaagcattccgaagccattgttagccgtatgaatagggaaactaaacccagtgataagacctgatgttttcgcttctttaattacatttggagattttttatttacagcattgttttcaaagatattccaattaattggtgaatgattggagttagaataatctactataggatcatattttattaaattagcgtcatcataatattgcctccattttttagggtaattatctagaattgaaatatcagatttaaccatagaatgaggataaatgatcgcgagtaaataatattcacaatgtaccattttagtcatatcagataagcattgattaatatcattattgcttctacaagctttaattttattaattatcctgtaagtgtcgtcggcatttatgtctttcatacccatctctttatccttacctattgtttgtcgcaagttttgcgtgttatatatcattaaaacggtaatggattgacatttgattctaataaattggatttttgtcacactattgtatcgctgggaatacaattacttaacataagtacctgtaggatcgtacaggtttacgcaagaaaatggtttgttatagtcgactaaacgcaagggaggttggtatgactataatgataaaaaaatcggattttttggcaattccatcggaggagtataaaggtattctaagtcttcgttatcaagtgtttaagcaaagacttgagtgggacttagttgtagaaaataaccttgnatcagangagtatgataactcaaatgcagaatata

SEQ ID NO: 15 - DNA, 1097 bp, Vibrio parahaemolyticus
  misc_feature (77)..(77): n is a, c, g, or t
tatttttgaaatatcccaagagctctttccttcgcatgcccacgctaaacattctttttctcttttggttaaatcgntgtttgatttattatttgctatatttatttttcgataattatcaactagagaaggaacaattaatggtatgttcatacacgcctgtaaaaataaactatctatatagttgtctttctctgaatgtgcaaaactaagcattccgaagccattattagccgtatgaatagggaaactaaacccagtgataagacctgatgatttcgcttctttaattacatttggagattttttatttacagcattgttttcaaatatattccaattaattggtgaatgattggagttagaataatctactataggatcatattttattaaattagcgtcatcataatattgcctccattttttagggtaattatctacaattgaaatatcagatttaaccatgcaatgaggataaatgatcgcgagtaaataatattcacaatgtaccattttagtcatatcagataagcattgattaatatccttattgcttctacagcgtttaattttattaattattctgtaagtgtcgtcggcatttatgtctttcatacccatctctttatccttacctattgtttgtcgcaagttttgcgtgttatatatcattaaaacggtaatggattggcatttgattctaataaattggatttttgtcacacctattgtatcgccaggagtacaattgattaacataagtaccctgtaggatcgtacaggtttacgcaagaaaatggtttgttatagtcgattaaacgcaagggaggttggtatgactataatgataaaaaaatcggattttttggcaattccatcggaggagtataaaggtattctaagtcttcgttatcaagtgtttaagcaaagacttgagtgggacttagttgtagaaaataaccttgaatcagatgagtatgataactcaaatgcagaatatatttatgcttgtgatgatactgaaaatgtaagtggatgctggcgtttattacctacaacaggtgattatatgctgaagaatgtttttcctgaat

SEQ ID NO: 16 - DNA, 6960 bp, Vibrio fischeri
gaattcttctttagaaatctgccggtaaaaattagattgctattcaatctatttctatcggtatttgtgaaataatactcaggataataatttacataaatattatcacgcattagagaagagcatgacttttttaatttaaacttttcattaacaaatcttgttgatatgaaaattttcctttgctattttaacagatattaaaacgggaataggcgttatattgacgatccattcagttagattaaaaaccttgagcagaaaatttatattattatcataattatgacgaaagttacaggccaggaaccacgtagtcagaatctgattttctatatatttgttatttacatcgtcataacacaaaaatataagaagcaagtgttggtacgaccagttcgcaagatagttaaacagcaacttaagttgaaattacccccattaaatggatggcaaatatgactaaaaaaatttcattcattattaacggccaggttgaaatctttcccgaaagtgatgatttagtgcaatccattaattttggtgataatagtgtttacctgccaatattgaatgactctcatgtaaaaaacattattgattgtaatggaaataacgaattacggttgcataacattgtcaattttctctatacggtagggcaaagatggaaaaatgaagaatactcaagacgcaggacatacattcgtgacttaaaaaaatatatgggatattcagaagaaatggctaagctagaggccaattggatatctatgattttatgttctaaaggcggcctttatgatgttgtagaaaatgaacttggttctcgccatatcatggatgaatggctacctcaggatgaaagttatgttcgggcttttccgaaaggtaaatctgtacatctgttggcaggtaatgttccattatctgggatcatgtctatattacgcgcaattttaactaagaatcagtgtattataaaaacatcgtcaaccgatccttttaccgctaatgcattagcgttaagttttattgatgtagaccctaatcatccgataacgcgctctttatctgttatatattggccccaccaaggtgatacatcactcgcaaaagaaattatgcgacatgcggatgttattgtcgcttggggagggccagatgcgattaattgggcggtagagcatgcgccatcttatgctgatgtgattaaatttggttctaaaaagagtctttgcattatcgataatcctgttgatttgacgtccgcagcgacaggtgcggctcatgatgtttgtttttacgatcagcgagcttgtttttctgcccaaaacatatattacatgggaaatcattatgaggaatttaagttagcgttgatagaaaaacttaatctatatgcgcatatattaccgaatgccaaaaaagattttgatgaaaaggcggcctattctttagttcaaaaagaaagcttgtttgctggattaaaagtagaggtggatattcatcaacgttggatgattattgagtcaaatgcaggtgtggaatttaatcaaccacttggcagatgtgtgtaccttcatcacgtcgataatattgagcaaatattgccttatgttcaaaaaaataagacgcaaaccatatctatttttccttgggagtcatcatttaaatatcgagatgcgttagcattaaaaggtgcggaaaggattgtagaagcaggaatgaataacatatttcgagttggtggatctcatgacggaatgagaccgttgcaacgattagtgacatatatttctcatgaaaggccatctaactatacggctaaggatgttgcggttgaaatagaacagactcgattcctggaagaagataagttccttgtatttgtcccataataggtaaaagtatggaaaatgaatcaaaatataaaaccatcgaccacgttatttgtgttgaaggaaataaaaaaattcatgtttgggaaacgctgccagaagaaaacagcccaaagagaaagaatgccattattattgcgtctggttttgcccgcaggatggatcattttgctggtctggcggaatatttatcgcggaatggatttcatgtgatccgctatgattcgcttcaccacgttggattgagttcagggacaattgatgaatttacaatgtctataggaaagcagagcttgttagcagtggttgattggttaactacacgaaaaataaataacttcggtatgttggcttcaagcttatctgcgcggatagcttatgcaagcctatctgaaatcaatgcttcgtttttaatcaccgcagtcggtgttgttaacttaagatattctcttgaaagagctttagggtttgattatctcagtctacccattaatgaattgccggataatctagattttgaaggccataaattgggtgctgaagtctttgcgagagattgtcttgattttggttgggaagatttagcttctacaattaataacatgatgtatcttgatataccgtttattgcttttactgcaaataacgataattgggtcaagcaagatgaagttatcacattgttatcaaatattcgtagtaatcgatgcaagatatattctttgttaggaagttcgcatgacttgagtgaaaatttagtggtcctgcgcaatttttatcaatcggttacgaaagccgctatcgcgatggataatgatcatctggatattgatgttgatattactgaaccgtcatttgaacatttaactattgcgacagtcaatgaacgccgaatgagaattgagattgaaaatcaagcaatttctctgtcttaaaatctattgagatattctatcactcaaatagcaatataaggactctctatgaaatttggaaactttttgcttacataccaacctccccaattttctcaaacagaggtaatgaaacgtttggttaaattaggtcgcatctctgaggagtgtggttttgataccgtatggttactggagcatcatttcacggagtttggtttgcttggtaacccttatgtcgctgctgcatatttacttggcgcgactaaaaaattgaatgtaggaactgccgctattgttcttcccacagcccatccagtacgccaacttgaagatgtgaatttattggatcaaatgtcaaaaggacgatttcggtttggtatttgccgagggctttacaacaaggactttcgcgtattcggcacagatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagcttcgacgactgagtgggctgctcaatttggcctaccgatgatattaagttggattataaatactaacgaaaagaaagcacaacttgagctttataatgaagtggctcaagaatatgggcacgatattcataatatcgaccattgcttatcatatataacatctgtagatcatgactcaattaaagcgaaagagatttgccggaaatttctggggcattggtatgattcttatgtgaatgctacgactatttttgatgattcagaccaaacaagaggttatgatttcaataaagggcagtggcgtgactttgtattaaaaggacataaagatactaatcgccgtattgattacagttacgaaatcaatcccgtgggaacgccgcaggaatgtattgacataattcaaaaagacattgatgctacaggaatatcaaatatttgttgtggatttgaagctaatggaacagtagacgaaattattgcttccatgaagctcttccagtctgatgtcatgccatttcttaaagaaaaacaacgttcgctattatattagctaaggagaaagaaatgaaatttggattgttcttccttaacttcatcaattcaacaactgttcaagaacaaagtatagttcgcatgcaggaaataacggagtatgttgataagttgaattttgaacagattttagtgtatgaaaatcatttttcagataatggtgttgtcggcgctcctctgactgtttctggttttctgctcggtttaacagagaaaattaaaattggttcattaaatcacatcattacaactcatcatcctgtcgccatagcggaggaagcttgcttattggatcagttaagtgaagggagatttattttagggtttagtgattgcgaaaaaaaagatgaaatgcatttttttaatcgcccggttgaatatcaacagcaactatttgaagagtgttatgaaatcattaacgatgctttaacaacaggctattgtaatccagataacgatttttatagcttccctaaaatatctgtaaatccccatgcttatacgccaggcggacctcggaaatatgtaacagcaaccagtcatcatattgttgagtgggcggccaaaaaaggtattcctctcatctttaagtgggatgattctaatgatgttagatatgaatatgctgaaagatataaagccgttgcggataaatatgacgttgacctatcagagatagaccatcagttaatgatattagttaactataacgaagatagtaataaagctaaacaagagacgcgtgcatttattagtgattatgttcttgaaatgcaccctaatgaaaatttcgaaaataaacttgaagaaataattgcagaaaacgctgtcggaaattatacggagtgtataactgcggctaagttggcaattgaaaagtgtggtgcgaaaagtgtattgctgtcctttgaaccaatgaatgatttgatgagccaaaaaaatgtaatcaatattgttgatgataatattaagaagtaccacatggaatatacctaatagatttcgagttgcagcgaggcggcaagtgaacgaatccccaggagcatagataactatgtgactggggtgagtgaaagcagccaacaaagcagcagcttgaaagatgaagggtataaaagagtatgacagcagtgctgccatactttctaatattatcttgaggagtaaaacaggtatgacttcatatgttgataaacaagaaattacagcaagctcagaaattgatgatttgattttttcgagcgatccattagtgtggtcttacgacgagcaggaaaaaatcagaaagaaacttgtgcttgatgcatttcgtaatcattataaacattgtcgagaatatcgtcactactgtcaggcacacaaagtagatgacaatattacggaaattgatgacatacctgtattcccaacatcggtttttaagtttactcgcttattaacttctcaggaaaacgagattgaaagttggtttaccagtagcggcacgaatggtttaaaaagtcaggtggcgcgtgacagattaagtattgagagactcttaggctctgtgagttatggcatgaaatatgttggtagttggtttgatcatcaaatagaattagtcaatttgggaccagatagatttaatgctcataatatttggtttaaatatgttatgagtttggtggaattgttatatcctacgacatttaccgtaacagaagaacgaatagattttgttaaaacattgaatagtcttgaacgaataaaaaatcaagggaaagatctttgtcttattggttcgccatactttatttatttactctgccattatatgaaagataaaaaaatctcattttctggagataaaagcctttatatcataaccggaggcggctggaaaagttacgaaaaagaatctctgaaacgtgatgatttcaatcatcttttatttgatactttcaatctcagtgatattagtcagatccgagatatatttaatcaagttgaactcaacacttgtttctttgaggatgaaatgcagcgtaaacatgttccgccgtgggtatatgcgcgagcgcttgatcctgaaacgttgaaacctgtacctgatggaacgccggggttgatgagttatatggatgcgtcagcaaccagttatccagcatttattgttaccgatgatgtcgggataattagcagagaatatggtaagtatcccggcgtgctcgttgaaattttacgtcgcgtcaatacgaggacgcagaaagggtgtgctttaagcttaaccgaagcgtttgatagttgatatcctttgcctaattgtaagtggaatgcttgcgttatataaatctgaatgacatctacactttacaaaattctccaaaacatccacatttgggtacttgatagaggtttatggggttggcttaacattgttctcattgttattattggctcaaagcaaaaggagataacatgaaaaaattggcagttatgcttgcattgggaatgattagctttggtgcaatggcagttgatgggtataaagatgcaaagtttggcatgacagaagaagagtttctttcgaagaggttatgtgattttgaaaaatttgagggagattctcgaatagaagaagtatcactttattcatgttctgacttttcgtttgctaacaaaaagcgtgaagcaatggcattttttttaaatgggaaatttaaaagattagagattaatattggcagacttgtgaagccagtaagcaaatcgttaacgaaaaagtacggagatggatcatcgtatccatcaaaagaagaatttgagaacgcgctaaaatacaatggaactatgtctataggttatgataataatacggtattagttgatatacatataatatgtggcaaagaaggcatagaaaccagtcaactgatttatacgagtccagatgtttatacgctcccagatttcggagaaaaaatccaggaattaaagggattaaaggaattc

SEQ ID NO: 17 - protein, 238 aa, Artificial sequence
  green fluorescent protein
MSKGEELFTGIVPVLIELDGDVHGHKFSVRGEGEGDADYGKLEIKFICTTGKLPVPWPTLVTTLGYGIQCFARYPEHMKMNDFFKSAMPEGYIQERTIFFQDDGKYKTRGEVKFEGDTLVNRIELKGMDFKEDGNILGHKLEYNFNSHNVYIMPDKANNGLKVNFKIRHNIEGGGVQLADHYQTNVPLGDGPVLIPINHYLSLQTAISKDRNETRDHMVFLEFFSACGHTHGMDELYK

SEQ ID NO: 18 - protein, 225 aa, Artificial sequence
  red fluorescent protein
MASSENVITEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVATVTQDSSLQDGCFIYKVKFIGVNFPSDGPVMQKKTMGWEASTERLYPRDGVLKGETHKALKLKDGGHYLVEFKSIYMAKKPVQLPGYYYVDAKLDITSHNEDYTIVEQYERTEGRHHLFL

SEQ ID NO: 19 - protein, 238 aa, Artificial sequence
  orange fluorescent protein
MSKGEELFTGVVPILVELDGDVHGHKFSVRGEGEGDADYGKLEIKFICTTGKLPVPWPTLVTTLGYGILCFARYPEHMKMNDFFKSAMPEGYIQERTIFFQDDGKYKTRGEVKFEGDTLVNRIELKGMDFKEDGNILGHKLEYNFNSHNVYIMPDKANNGLKVNFKIRHNIEGGGVQLADHYQTNVPLGDGPVLIPINHYLSYQTAISKDRNETRDHMVFLEFFSACGHTHGMDELYK

SEQ ID NO: 20 - protein, 489 aa, Artificial sequence
  Alkaline phosphatase
MKQSTIALALLPLLFTPVTKARTPEMPLQGTAVDGGGGSMHASLEVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 21 - protein, 309 aa, Artificial sequence
  Horseradish peroxidase
MQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNS

SEQ ID NO: 22 - protein, 286 aa, Artificial sequence
  His tag
QSVLTQPPSVSGSPGQSITISCTGTSSDIGTYKIVSWYQQHPGKAPKLMIYDVNQRPSGVSDRFSGSKSGNTASLTISGLQAEDEADYYCSSYTSGSTLVFGGGTKLTVLGSSGGGGSGGGGSGGSALQVQLQQSGAEVKKPGESLKISCKGSGYSFTSYWIGWVRQMPGKGLEWMGIIYPGDSDTRYSPSFQGQVTISADKSISTAYLQWSSLKASDTAMYYCARHRAASGSPDACDYWGQGTLVTVSSGSASAPTLFPAAAHHHHHHGAAEQKLISEEDLNGAA

SEQ ID NO: 23 - protein, 286 aa, Artificial sequence; identical to SEQ ID NO: 22
  Myc tag

SEQ ID NO: 24 - protein, 15 aa, Artificial sequence
  Biotin lygase tag
LHHILDAQKMVWNHR

SEQ ID NO: 25 - protein, 1019 aa, Artificial sequence
  Beta galactosidase
MADPVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSADFQLSAGRYHYQLVWCQK

SEQ ID NO: 26 - protein, 159 aa, Artificial sequence
  Streptavidin
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 27 - DNA, 717 bp, Artificial sequence
  Oligonucleotide encoding green fluorescent protein
atgagtaaaggagaagaacttttcactgggattgtcccagttctcattgagttagacggtgatgtccatggacataaattctctgtcagaggagaaggggaaggcgatgcagattatggaaaacttgaaatcaaattcatttgcactactggaaagctaccagttccatggccaacacttgttactacactgggctacggcatccaatgtttcgcaagatacccagaacacatgaaaatgaatgacttcttcaagagtgccatgcctgagggttacattcaagaaagaaccatctttttccaagatgatggaaaatacaagacacgtggtgaagtcaagtttgaaggtgatactcttgttaacagaattgagctcaaaggtatggactttaaagaagatggcaatatccttggacacaagttggagtacaattttaattcacataatgtatacattatgccggacaaagccaataatggactcaaagtcaatttcaaaattagacacaatatcgaaggtggtggtgtccaacttgctgatcattaccaaacaaatgttccccttggagacggtcctgtccttataccaatcaatcactacctatccttgcaaacagccatttcaaaagatcgaaatgagacgagagatcatatggtgtttctggaatttttctcagcttgtggacatacacatggcatggatgaactatacaaataa

SEQ ID NO: 28 - DNA, 2861 bp, Artificial sequence
  Oligonucleotide encoding red fluorescent protein
ctcgaggatcccgggtacctgcagctagcgtcgacagaggaattaaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtaggcggccgctctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 29 - DNA, 717 bp, Artificial sequence
  Oligonucleotide encoding orange fluorescent protein
atgagtaaaggagaagaacttttcactggagttgtcccaattcttgttgaattagatggtgatgtccatggacataaattctctgtcagaggagaaggggaaggcgatgcagattatggaaaacttgaaatcaaattcatttgcactactggaaagctaccagttccatggccaacacttgttactacactgggctatggcatcctatgtttcgcaagatacccagaacacatgaaaatgaatgacttcttcaagagtgccatgcctgagggttacattcaagaaagaaccatctttttccaagatgatggaaaatacaagacacgtggtgaagtcaagtttgaaggtgatactcttgttaacagaattgagctcaaaggtatggactttaaagaagatggcaatatccttggacacaagttggagtacaattttaactcacataatgtatacattatgccggacaaagccaataatggactcaaagtcaatttcaaaattagacacaatatcgaaggtggtggtgtccaactcgctgatcattaccaaacaaatgttccccttggagacggtcctgtccttataccaatcaatcactacctatcctatcaaacagccatttcaaaagatcgaaatgagacgagagatcatatggtgtttctggaatttttctcagcttgtggacatacacatggcatggatgaactatacaaataa

SEQ ID NO: 30 - DNA, 7151 bp, Artificial sequence
  Oligonucleotide encoding Alkaline phosphatase
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttttttcgccaggcgcagacttgctgttcttcaggcaatcactcatgtaggtcttacgagcatcccctttcaacgcctgcgccgtcgcctgctgattacaggaggtcatacgctgttgttgtggggttaaagttctctcggcagcgccgacggtggttaaaaaaaccagaccgaaaagcaaggtaaccagtaatgttattttcatagcaccatccctcttcatgttttaaccatgagcgtatgcgcccgtgatctgccattaagtctggttgctaacagcaaaaaaaccacccggcagcgaaaattcactgccgggcgcggttttatttcagccccagagcggctttcatggtgtagaagagatcggtctggtcggtcagtccaacaacattggcggcatgcgggccatacgccgcaatacgcaactgactgccggtatgttcttgtgaatcctcttcggagttcccgtaactcatcaccatcactgcgccatctttggtatttagcgcctgggtgaggcccggagctttggtatccggcgcaacaatctggctggcgtgggcgtgatcagcggtgactatgaccagcgtgttaccctcctttttagcgaattccagcgcccgttgtacggcttcatcgagatcgaccgtctcgccaatttgcccacaaggattcgcagcatgatcctgtttatcgattgacgcaccttcaacttgcaggaaaaagcctttctcatttttactcaacaattcaatggctttgtcggtcatctgcgccagggttggtacactgtcattacgttgcggatttggcgtacaggtgactgcgggcttatcgatattgccatggtacgttgctttcggtcctagccagcgcactggcatattgccgtcagcaaacaggccaagcaggggtttttgctgattcgcttccgtcaccgaattcagtgaggcagcatcgctcaccaactgataaccacgcgcctgtgcctgttcacgcagcgtttttccctgccattcaccagcggttgccgtttcagcaaaggtttttgcgccgccgccaagcgtaacgtcggcacgagcgttaagcagctgttcggtaatcgatccttttccgcctttttccagagcgttacccggacatttttcactggtcgcgctcggaccgtagcatttgcgcgaggtcacatgtgccaccagcgcagcgggcgtggcatcctgcaactctgcggtagaaacgttaccggtcgccagacctgcggcttttgccatttccagaatcgttgggtgatctttttcgtgaatatcgacgcccagcgcgccgttataggttttgacaccggttgaccaggcggttgctgatgcagccgagtcggtgacgtagtccggtttgccggtttttttattcagcgcatagtgagtgtattgcccggtaagcggtaaggcatctatacctttaaaaaagccgcccgcaccttcggcataattacgtgcggcagtaatttccgagtcccccatcccatcgccaatcagcaaaataatattttttgcaggtttatcgctaagagaatcacgcagagcggcagtctgatcacccgttaaacggcgagcaccgccgggtgcagtaatatcgccctgagcagcccggttttccagaacctcgaggctagcatgcatagaaccgccaccaccgtcgacagcggtaccctgcagaggcatttctggtgtccgggcttttgtcacaggggtaaacagtaacggtaagagtgccagtgcaatagtgctttgtttcactttattttctccatgtcgcgtcttatcagggggaattctgtttcctgtgtgaaattgttatccgctcacaattccacacattatacgagccgatgattaattgtcaacagctcatttcagaatatttgccagaaccgttatgatgtcggcgcaaaaaacattatctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 31 - DNA, 955 bp, Artificial sequence
  Oligonucleotide encoding Horseradish peroxidase
aagcttaaccatgcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcctagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatctgcggaccccaaccatcttcgataacaagtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactcttaataaggatccgaattc

SEQ ID NO: 32 - DNA, 861 bp, Artificial sequence
  ynthetic construct SP-1A2 recombinant single chain Fv antibody mRNA
cagtctgtgttgacgcagccgccctcagtgtctgggtctcctggacagtcgatcaccatctcctgcactggaaccagcagtgatattgggacttataaaattgtctcctggtaccaacagcaccctggcaaagcccccaaactcatgatttatgacgtcaatcagcggccctcaggggtttctgatcgcttctctggctccaagtctggcaacacggcctccctgacaatctctgggctccaggctgaggacgaggctgattattactgcagctcatatacaagcggcagcactctggtattcggcggggggaccaagctgaccgtcctaggctcgagtggtggaggcggttcaggcggaggtggctctggcggtagtgcacttcaggtacagctgcagcagtcaggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacatcgggccgctagtgggagcccggacgcgtgtgactactggggccagggaaccctggtcaccgtctcctcagggagtgcatccgccccaacccttttccccgcggccgcacatcatcatcaccatcacggggccgcagaacaaaaactcatctcagaagaggatctgaatggggccgcatag

SEQ ID NO: 33 - DNA, 861 bp, Artificial sequence; identical to SEQ ID NO: 32
  synthetic construct SP-1A2 recombinant single chain Fv antibody mRNA

SEQ ID NO: 34 - DNA, 8388 bp, Artificial sequence
  Oligonucleotide encoding Beta galactosidase
acgttaagggattttggtcatggacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgatatgcgtaaggagaaaataccgcatcaggcgcatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaattaaggaatcccccaggacccaacgctgcccgagtttgtcagaaagcagaccaaacagcggttggaataatagcgagaacagagaaatagcggcaaaaataatacccgtatcacttttgctgatatggttgatgtcatgtagccaaatcgggaaaaacgggaagtaggctcccatgataaaaaagtaaaagaaaaagaataaaccgaacatccaaaagtttgtgttttttaaatagtacataatggatttccttacgcgaaatacgggcagacatggcctgcccggttattattatttttgacaccagaccaactggtaatggtagcgaccggcgctcagctggaaatccgccgatactgacgggctccaggagtcgtcgccaccaatccccatatggaaaccgtcgatattcagccatgtgccttcttccgcgtgcagcagatggcgatggctggtttccatcagttgctgttgactgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggcggcagtaaggcggtcgggatagttttcttgcggccctaatccgagccagtttacccgctctgctacctgcgccagctggcagttcaggccaatccgcgccggatgcggtgtatcgctcgccacttcaacatcaacggtaatcgccatttgaccactaccatcaatccggtaggttttccggctgataaataaggttttcccctgatgctgccacgcgtgagcggtcgtaatcagcaccgcatcagcaagtgtatctgccgtgcactgcaacaacgctgcttcggcctggtaatggcccgccgccttccagcgttcgacccaggcgttagggtcaatgcgggtcgcttcacttacgccaatgtcgttatccagcggtgcacgggtgaactgatcgcgcagcggcgtcagcagttgttttttatcgccaatccacatctgtgaaagaaagcctgactggcggttaaattgccaacgcttattacccagctcgatgcaaaaatccatttcgctggtggtcagatgcgggatggcgtgggacgcggcggggagcgtcacactgaggttttccgccagacgccactgctgccaggcgctgatgtgcccggcttctgaccatgcggtcgcgttcggttgcactacgcgtactgtgagccagagttgcccggcgctctccggctgcggtagttcaggcagttcaatcaactgtttaccttgtggagcgacatccagaggcacttcaccgcttgccagcggcttaccatccagcgccaccatccagtgcaggagctcgttatcgctatgacggaacaggtattcgctggtcacttcgatggtttgcccggataaacggaactggaaaaactgctgctggtgttttgcttccgtcagcgctggatgcggcgtgcggtcggcaaagaccagaccgttcatacagaactggcgatcgttcggcgtatcgccaaaatcaccgccgtaagccgaccacgggttgccgttttcatcatatttaatcagcgactgatccacccagtcccagacgaagccgccctgtaaacggggatactgacgaaacgcctgccagtatttagcgaaaccgccaagactgttacccatcgcgtgggcgtattcgcaaaggatcagcgggcgcgtctctccaggtagcgaaagccattttttgatggaccatttcggcacagccgggaagggctggtcttcatccacgcgcgcgtacatcgggcaaataatatcggtggccgtggtgtcggctccgccgccttcatactgcaccgggcgggaaggatcgacagatttgatccagcgatacagcgcgtcgtgattagcgccgtggcctgattcattccccagcgaccagatgatcacactcgggtgattacgatcgcgctgcaccattcgcgttacgcgttcgctcatcgccggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgcctcgaatcagcaacggcttgccgttcagcagcagcagaccattttcaatccgcacctcgcggaaaccgacatcgcaggcttctgcttcaatcagcgtgccgtcggcggtgtgcagttcaaccaccgcacgatagagattcgggatttcggcgctccacagtttcgggttttcgacgttcagacgtagtgtgacgcgatcggcataaccaccacgctcatcgataatttcaccgccgaaaggcgcggtgccgctggcgacctgcgtttcaccctgccataaagaaactgttacccgtaggtagtcacgcaactcgccgcacatctgaacttcagcctccagtacagcgcggctgaaatcatcattaaagcgagtggcaacatggaaatcgctgatttgtgtagtcggtttatgcagcaacgagacgtcacggaaaatgccgctcatccgccacatatcctgatcttccagataactgccgtcactccaacgcagcaccatcaccgcgaggcggttttctccggcgcgtaaaaatgcgctcaggtcaaattcagacggcaaacgactgtcctggccgtaaccgacccagcgcccgttgcaccacagatgaaacgccgagttaacgccatcaaaaataattcgcgtctggccttcctgtagccagctttcatcaacattaaatgtgagcgagtaacaacccgtcggattctccgtgggaacaaacggcggattgaccgtaatgggataggttacgttggtgtagatgggcgcatcgtaaccgtgcatctgccagtttgaggggacgacgacagtatcggcctcaggaagatcgcactccagccagctttccggcaccgcttctggtgccggaaaccaggcaaagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacgggatcagccatttttttctccttacttacttaggatccccgggtaccgagctcgaattggggatcttgaagttcctattccgaagttcctattctctagaaagtataggaacttcagagcgcttttgaagctaattcgagctcggtacccggggatcccccgggctcgactgcattaatgaatcggccaacgcgcggggagaggcggtttgcgtattgggcgctcttccgctcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtactcgagcagaaagtcaaaagcctccgaccggaggcttttgacttgagggggatcgatcccttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccggcgtcgtgatcgccgccgagaatgcccttcaccaagttcgacgacacgaaaatcatgctgacggctatcaccatcatgcagacggatcgcacgaacccgcagaactcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgccgcgatgaggaagcgggtgccccgcttcttcatcttcgcgcctcgggcctccaggccgcctacctgggcgaaaacatcggtgtttgtggcattcatacggactcctgttgggccagctcgcgcacgggctggcgggtcagcttggcttgaagatcgccacgcattgcggcgatctgcttctcggcatccttgcgcttctgcacgccttcctgctggatgcgaataacgtcctcgacggtcttgatgagcgtcgtctgaacctgcttgagcgtgtccacgtcgatcaccaggcgttggttctccttcgccgtctcgacggacgtgcgatgcagcagggccgcattgcgcttcatcaggtcgttggtggtgtcgtcgatggccgtggccagttcgacggcgttcttctgctcgttgaggctcaaggccagcatgaattgccgcttccacgccggcacggtgatttcgcggatggtgtggaatttatcgaccagcatctggttgttggcctggatcatgcggatggtcggcaggctctgcatggccgaatgttgcaaggcgatcaggtcgccgatgcgcttgtccaggttggcaaccatcgcatcgaggtcggccagctcctgcacgcggccagggtcgttcccgacattgccgcgcagaccctcggcctgctcgcgcagctcggcaaggcggaccttgccggccgcgatgtggacgccaagaaggcggtgttcctcgcgcacggctgcgaacatttcgtcgagcgaggcattgcgctgcgcgatgccttgctgggtggtctgcacttcgctgaccaggtgttcgatctgctcgcgggtcgtgtcgaagcgcgccatgaagcccgtcgaacggacgcggaagcggtcgatcagcgggccaatcaggggcaggcgggaacggttgtcggacaaagggccgacgttcagggaacgggccttggcgacaacctgggtcagtttctcgcctgcttcgtccaggtcgctgttgcgcacctggtccagcaggctatcggcgtagcgggacgtgtgctcggccacgtcgcggccgaactcggcaacggtctgcggactgccgacctcgatccgctgcgcgaccgcatggacttccggcacgtcgctttcctgcaagcccagctcgcgcagggttgccggggtcatgtcgaaggcgacgataggggccttggcgtcgtgcgtcgttttcagtgcgttcatagggttctcccgccgtgttattggttgatgccttccaggctctgcgaaaggctccgcatgagcgcctggtgagctttggccgcctcggcgaccattgccggattcatgttcttggtggtgatgagcgcgagggtgtgctgacgccagacgggcaccaggacggatgccgtttcagagaagcggtccagcatgtccacggcctgcgcccgcgtgagcttcatctgagtgacgctcatttcatgggacgccatgagggttgccaggttggcgagcttgcgcgcgaagcgttcgcgcggcttgtcgaactcgatcacgccggccttggccgcgccggcctcggggttctcgtccaggaactcgcgcccggcttgaatgtaggctctgagccggtctacctcggcctcatgcgtattgagcatgtcatccaaggcgcgcaacgtgtcccgcacgcgctgcgctacgccctcggcttcgtccagcaactggtcgagcgtcttgcgggcgacctgatacctcacctggcgttcaacctcacggccaagcatcttctcgaaccaggtaggcttttccgcgatcttgcgggggtccgcgtcggccagcttcgccacgatctggctgattttgtcggccagcgcggcaactgcgccgtgctccatcagattcgacagctcgttgagggaatccgccccgtcgatgccggccccgtactcgccaatcgtcgccggcgacgcgaagagggcgggcaaaacctcccccttcaatcgcgccatgttcacgctttgttcttccatggtatatctccttcttaaagttaaacaaaattattcggaacccagcatgatattccggaaataccaactaagtcaacggctgatggccaattcggcttcctcgctcactgactcgctgcgctcggtcgttcggctgcggcgagcggtatcagctcactcaaaggcggtaatacggttatccacagaatcaggggataacgcaggaaagaacatgtgagatcgatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagctagcttttaaggtttaacggttgtggacaacaagccagggatgtaacgcactgagaagcccttagagcctctcaaagcaattttcagtgacacaggaacacttaacggctgacatgacgctcagtggaacgaaaactc

SEQ ID NO: 35 - DNA, 483 bp, Artificial sequence
  Oligonucleotide encoding Streptavidin
gacccgagcaaagattctaaagcacaagtatctgctgcagaagcaggaattacaggcacatggtataatcagctgggatctacatttattgttacagccggcgcagatggagctcttacaggaacatatgaatctgctgttggaaatgcagaatctagatacgtgcttacaggaagatatgattctgcacctgcaacagatggatccggaacagcacttggatggacagttgcatggaaaaacaattatagaaacgcacatagcgctacaacatggtctggccaatatgtgggaggtgcagaagcaagaattaacacacaatggcttttaacatctggaacaacagaagcaaatgcatggaaaagtactcttgttggacatgatacatttacaaaagttaaacctagcgcagcatctatcgatgcagcgaaaaaagcaggagttaacaatggcaatcctttagatgcagttcaacaataatga

SEQ ID NO: 36 - DNA, 20 bp, Artificial sequence
  Oligonucleotide encoding V fischeri lux box
acctgtacgatcctacaggt

SEQ ID NO: 37 - DNA, 18 bp, Artificial sequence
  Oligonucleotide encoding Agrobacterium tumefaciens tra box
acgtgcagatctgcacat

SEQ ID NO: 38 - DNA, 18 bp, Artificial sequence
  Oligonucleotide encoding Pseudomonas aeruginosa rhl box
ctgccagatttcacagga

SEQ ID NO: 39 - DNA, 20 bp, Artificial sequence
  Qsc102
acctgcccggaagggcaggt

SEQ ID NO: 40 - DNA, 20 bp, Artificial sequence
  Qsc117
cactgccagatctggcagtt

SEQ ID NO: 41 - DNA, 20 bp, Artificial sequence
  phzA
acctaccagatcttgtagtt

SEQ ID NO: 42 - DNA, 18 bp, Artificial sequence
  Oligonucleotide encoding Burkholderia cenocepacia cep box
ctgtaagagttaccagtt

SEQ ID NO: 43 - DNA, 71 bp, Artificial sequence
  Oligonucleotide encoding V fischeri lux box Lux box +promoter
acctgtaggatcgtacaggtttacgcaagaaaatggtttgttatagtcgaataaacgcaagggaggttggt

SEQ ID NO: 44 - protein, 229 aa, Artificial Sequence
  Amcyan
MALSNKFIGDDMKMTYHMDGCVNGHYFTVKGEGSGKPYEGTQTSTFKVTMANGGPLAFSFDILSTVFMYGNRCFTAYPTSMPDYFKQAFPDGMSYERTFTYEDGGVATASWEISLKGNCFEHKSTFHGVNFPADGPVMAKMTTGWDPSFEKMTVCDGILKGDVTAFLMLQGGGNYRCQFHTSYKTKKPVTMPPNHAVEHRIARTDLDKGGNSVQLTEHAVAHITSVVPF

SEQ ID NO: 45 - DNA, 2875 bp, Artificial Sequence
  Reporter gene-fusion vector pFU81
ctcgaggatcccgggtacctgcagctagcgtcgacaggaggaattaaccatggctctttcaaacaagtttatcggagatgacatgaaaatgacctaccatatggatggctgtgtcaatgggcattactttaccgtcaaaggtgaaggcagcgggaagccatacgaagggacgcagacctcgacttttaaagtcaccatggccaacggtgggccccttgcattctcctttgacatactatctacagtgttcatgtatggaaatcgatgctttactgcgtatcctaccagtatgcccgactatttcaaacaagcatttcctgacggaatgtcatatgaaaggacttttacctatgaagatggaggagttgctacagccagttgggaaataagccttaaaggcaactgctttgagcacaaatccacgtttcatggagtgaactttcctgctgatggacctgtgatggcgaagatgacaactggttgggacccatcttttgagaaaatgactgtctgcgatggaatattgaagggtgatgtcaccgcgttcctcatgctgcaaggaggtggcaattacagatgccaattccacacttcttacaagacaaaaaaaccggtgacgatgccaccaaaccatgcggtggaacatcgcattgcgaggaccgaccttgacaaaggtggcaacagtgttcagctgacggagcacgctgttgcacatataacctctgttgtccctttctgaagcggccgctctagaggcatcaaataaaacgaaaggctcagtcgaaagactgggcctttcgttttatctgttgtttgtcggtgaacgctctcctgagtaggacaaatccgccgccctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 46 - DNA, 20 bp, Artificial Sequence
  Oligonucleotide encoding Pseudomoans aeruginosa las box
atctatctcatttgctagtt